SEQUENCE LISTING (6 sequences)

SEQ ID NO: 1 - protein, 402 aa, Artificial sequence
  IL-10-Fc monomer
EPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGPGSGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 421 aa, Artificial sequence
  IL-10-Fc monomer with a leader peptide
MMSFVSLLLVGILFHATQAEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGSGGGPGSGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 3 - DNA, 1206 bp, Artificial sequence
  DNA IL-10-Fc
gagcccaaatctagtgacaaaactcacacatgcccaccgtgcccagcacctgaagccgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaaaagcctctccctgtccccgggtggagggtcaggaggtggtccaggatcgggtggctctcctggacagggaacccagtccgagaactcctgcacccacttccccggcaacctgcccaacatgctgcgggacctgagagatgccttctccagagtgaaaacattcttccagatgaaggaccagctggacaacctgctgctgaaagagtccctgctggaagatttcaagggctacctgggctgccaggccctgtccgagatgatccagttctacctggaagaagtgatgcctcaggccgagaaccaggaccccgacatcaaggcccacgtgaactccctgggcgagaacctgaaaaccctgcggctgcggctgagaagatgccaccggtttctgccctgcgagaacaagtccaaggccgtggaacaagtgaagaacgccttcaacaagctgcaggaaaagggcatctacaaggccatgagcgagttcgacatcttcatcaactacatcgaggcctacatgaccatgaagatccggaac

SEQ ID NO: 4 - DNA, 1206 bp, Artificial sequence
  DNA IL-10-Fc
gagcccaaatctagtgacaaaactcacacatgcccaccgtgcccagcacctgaagccgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaaaagcctctccctgtccccgggtggagggtcaggaggtggtccaggatcgggtggctcgcccggtcaaggaactcagtctgagaactcatgtacacactttccagggaatctgcccaacatgcttagggatctccgggacgctttctctagggtcaagaccttcttccagatgaaagaccaactggacaatctgctgttaaaggaaagtttgctcgaagattttaagggctatctcgggtgccaggccctttccgaaatgattcagttttacctggaggaagtaatgcctcaagctgaaaaccaggaccccgatatcaaggcgcatgtgaattcattgggcgagaacctgaaaaccctccgactcagactccggcgttgccacaggttcctgccatgcgagaacaaatcaaaggccgtcgagcaggtgaagaacgcattcaataagctgcaggagaaaggaatctacaaagctatgtcggagttcgacatcttcatcaactatatcgaggcttacatgactatgaaaatccgtaat

SEQ ID NO: 5 - DNA, 1263 bp, Artificial sequence
  DNA IL-10-Fc with a leader peptide
atgatgtcctttgtctctctgctcctggttggcatcctgttccatgccacccaggccgagcccaaatctagtgacaaaactcacacatgcccaccgtgcccagcacctgaagccgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaaaagcctctccctgtccccgggtggagggtcaggaggtggtccaggatcgggtggctctcctggacagggaacccagtccgagaactcctgcacccacttccccggcaacctgcccaacatgctgcgggacctgagagatgccttctccagagtgaaaacattcttccagatgaaggaccagctggacaacctgctgctgaaagagtccctgctggaagatttcaagggctacctgggctgccaggccctgtccgagatgatccagttctacctggaagaagtgatgcctcaggccgagaaccaggaccccgacatcaaggcccacgtgaactccctgggcgagaacctgaaaaccctgcggctgcggctgagaagatgccaccggtttctgccctgcgagaacaagtccaaggccgtggaacaagtgaagaacgccttcaacaagctgcaggaaaagggcatctacaaggccatgagcgagttcgacatcttcatcaactacatcgaggcctacatgaccatgaagatccggaac

SEQ ID NO: 6 - DNA, 1263 bp, Artificial sequence
  DNA IL-10-Fc with a leader peptide
atgatgtcctttgtctctctgctcctggttggcatcctgttccatgccacccaggccgagcccaaatctagtgacaaaactcacacatgcccaccgtgcccagcacctgaagccgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaaaagcctctccctgtccccgggtggagggtcaggaggtggtccaggatcgggtggctcgcccggtcaaggaactcagtctgagaactcatgtacacactttccagggaatctgcccaacatgcttagggatctccgggacgctttctctagggtcaagaccttcttccagatgaaagaccaactggacaatctgctgttaaaggaaagtttgctcgaagattttaagggctatctcgggtgccaggccctttccgaaatgattcagttttacctggaggaagtaatgcctcaagctgaaaaccaggaccccgatatcaaggcgcatgtgaattcattgggcgagaacctgaaaaccctccgactcagactccggcgttgccacaggttcctgccatgcgagaacaaatcaaaggccgtcgagcaggtgaagaacgcattcaataagctgcaggagaaaggaatctacaaagctatgtcggagttcgacatcttcatcaactatatcgaggcttacatgactatgaaaatccgtaat

(19) United States
(12) Patent Application Publication
(10) Pub. No.: US 2024/0034764 A1
(43) Pub. Date: Feb. 1, 2024

(54) IMMUNOCYTOKINE FOR ACTIVATING HUMAN IL-10RA RECEPTOR AND USE THEREOF

(71) Applicant: JOINT STOCK COMPANY "BIOCAD", Saint Petersburg (RU)

(72) Inventors: Aleksey Vladimirovich KONONOV, g. Pushchino (RU); Iuliia Viktorovna EVDOKIMOVSKAIA, g. Pushchino (RU); Iana Andreevna SMIRNOVA, Moscow (RU); Stanislav Rudolfovich EVDOKIMOV, g. Pushchino (RU); Elena Sergeevna KOLOSOVA, g. Perm (RU); Sergei Andreevich AGEEV, g. Chehov (RU); Vladimir Sergeevich TSYMPILOV, s. Zakulta (RU); Valery Vladimirovich SOLOVYEV, g.Pushchino (RU); Pavel Andreevich IAKOVLEV, Saint Petersburg (RU); Dmitry Valentinovich MOROZOV, Saint Petersburg (RU)

(21) Appl. No.: 18/266,001

(22) PCT Filed: Dec. 10, 2021
(86) PCT No.: PCT/RU2021/050432

§ 371 Date: Jun. 8, 2023

(30) Foreign Application Priority Data
Dec. 10, 2020 (RU) 2020140807

(51) Int. Cl. C07K 14/54; C12N 15/63; A61P 35/00; C07K 16/28

(52) U.S. Cl. CPC C07K 14/5428; C12N 15/63; A61P 35/00; C07K 16/2818; C07K 2319/30

(57) ABSTRACT

The present invention relates to the field of biotechnology and medicine, in particular to an immunocytokine for activating the human IL-10Rα receptor. The invention further relates to nucleic acids encoding said immunocytokine, expression vectors, host cells and methods for producing same, methods for producing the immunocytokine, pharmaceutical compositions comprising the above immunocytokine, pharmaceutical compositions comprising the above immunocytokine and other therapeutically active compounds, methods for treating an oncological disease and the uses of the immunocytokine or pharmaceutical compositions thereof for treating an oncological disease.

### FIELD OF THE INVENTION

The present invention relates to the field of biotechnology and medicine, in particular to an immunocytokine for activating the human IL-10Rα receptor. The invention further relates to nucleic acids encoding said immunocytokine, expression vectors, host cells and methods for producing same, methods for producing the immunocytokine, pharmaceutical compositions comprising the above immunocytokine, pharmaceutical compositions comprising the above immunocytokine and other therapeutically active compounds, methods for treating an oncological disease and the uses of the immunocytokine or pharmaceutical compositions thereof for treating an oncological disease.

### BACKGROUND OF THE INVENTION

Interleukin 10 (or IL-10) is a cytokine and until recently was viewed as an anti-inflammatory cytokine. It is a homodimer with a molecular weight of approximately 37-39 kDa. Human and interleukin 10 has high homology with murine interleukin 10, which is about 80%.

IL-10 is able to abrogate angiogenesis, metastasis, and have a direct antitumor effect (Martin Oft, IL-10: Master Switch from Tumor-Promoting Inflammation to Antitumor Immunity, Cancer Immunology at the Crossroads: Experimental Immunotherapies, March 2014, Volume 2, Issue 3, 10.1158/2326-6066.CIR-13-0214).

It has been shown that one of the likely antitumor effects of IL-10 is its activating effect on PD-1-expressing CDS lymphocytes. IL-10 leads to potent antiapoptotic intracellular cascades, which fact contributes to the survival of cytotoxic lymphocytes in the tumor microenvironment (Martin Oft, Immune regulation and cytotoxic T cell activation of IL-10 agonists — Preclinical and clinical experience, Seminars in Immunology, Volume 44, August 2019, DOI:10.1016/j .smim.2019.101325).

IL-10 is characterized by a short plasma half-life due to its small size, which is approximately 37-39 kDa, resulting in rapid renal clearance; in fact, its half-life in the systemic compartment is 2.5 hours (Braat H. et al., Interleukin-10-based therapy for inflammatory bowel disease, Expert Opin. Biol. Ther. 3(5), 2003, pp. 725-731). Pegylation of this cytokine has been employed by some authors to increase retention time in the bloodstream, exposure, efficacy, and to reduce renal absorption (Mattos A. et al., PEGylation of interleukin-10 improves the pharmacokinetic profile and enhances the antifibrotic effectivity in CCl4-induced fibrogenesis in mice, J. Control Release 162, 2012, pp. 84-91 and Mumm J. B. et al., IL-10 elicits IFNγ-dependent tumor immune surveillance, Cancer Cell 20(6), 2011, pp. 781-796). However, the disadvantages of pegylation may include potential risks such as immunogenicity, decreased biological activity of the pegylated molecule and heterogeneity of pegylation, which fact may lead to the production of therapeutic molecules with different properties (size, charge, etc.), and the formation of a hydrophilic coating surrounding the therapeutic molecule. Furthermore, this approach can extend the half-life of the formulation in the bloodstream to no more than 24 hours.

Another method for extending the half-life of IL-10 in plasma is to use fusion proteins comprising an antibody or a fragment thereof and IL-10 for the directional transport of the cytokine to inflamed tissues and accumulation of the cytokine therein.

The prior art provides articles (Zheng, X. X. ET AL., A noncytolytic IL-10/Fc fusion protein prevents diabetes, blocks autoimmunity, and promotes suppressor phenomena in NOD mice. J. Immunol. 1997, 158, 4507-4513 ϰ Zheng, X. X. ET AL., Administration of noncytolytic IL-10/Fc in murine models of lipopolysaccharide-induced septic shock and allogeneic islet transplantation. J. Immunol. 1995, 154, 5590-5600), which describe an immunocytokine based on IL-10 and an Fc fragment.

International applications WO2012045334 and WO2014023673 further provide a fusion protein comprising a heterodimeric complex based on IL-10 and an Fc fragment.

International application WO2015117930 provides a fusion protein comprising an IgG-class antibody and a mutant IL-10 molecule, wherein the fusion protein comprises two identical heavy chain polypeptides and two identical light chain polypeptides, and wherein the mutant IL-10 molecule comprises an amino acid substitution that reduces the binding affinity of the mutant IL-10 molecule to the IL-10 receptor, as compared to a wild- type IL-10 molecule.

The above fusion proteins (immunocytokines) based on IL-10 and Fc fragment have a common disadvantage, in particular insufficient stability in the blood, since they are prone to degradation by serum proteases.

In connection with the above, there is a need to develop fusion proteins (immunocytokines) based on IL-10 and Fc fragment, which will show an extended half-life in blood plasma as compared to IL-10 and will be stable due to resistance to serum protease degradation.

### BRIEF DESCRIPTION OF THE INVENTION

As found by the authors, the immunocytokine comprising a homodimeric complex based on IL-10 and human IgG1 Fc fragment provided in the present invention has an extended plasma half-life as compared to that of IL-10 and is stable due to resistance to serum protease degradation thanks to the unique structure thereof.

In one aspect, the present invention relates to an immunocytokine for activating the human IL-10Rα receptor, which comprises a homodimeric complex based on IL-10 and human IgG1 Fc fragment, wherein the monomer based on IL-10 and human IgG1 Fc fragment comprises the amino acid sequence of SEQ ID NO: 1.

In one aspect, the present invention relates to an isolated nucleic acid that encodes the above immunocytokine.

In some embodiments, the isolated nucleic acid is DNA.

In some embodiments, the isolated nucleic acid includes the nucleotide sequence with SEQ ID NO:3.

In some embodiments, the isolated nucleic acid includes the nucleotide sequence with SEQ ID NO:4.

In one aspect, the present invention relates to an expression vector comprising any of the above nucleic acids.

In one aspect, the present invention relates to a method for production of a host cell for production of the above immunocytokine of the invention, which comprises transformation of the cell with the above vector.

In one aspect, the present invention relates to a host cell for production of the above immunocytokine of the invention, the host cell comprises any of the above nucleic acids.

In one aspect, the present invention relates to a method for preparing a formulation comprising the above immunocytokine of the invention, which comprises culturing of the above host cell in a culture medium under conditions sufficient to produce said immunocytokine, if necessary, followed by isolation and purification of the resulting immunocytokine of the invention.

In one aspect, the present invention relates to a pharmaceutical composition for activating the human IL-10Rα receptor, which comprises the above immunocytokine of the invention and one or more pharmaceutically acceptable excipients.

In one aspect, the present invention relates to a pharmaceutical composition for activating the human IL-10Rα receptor, which comprises the above immunocytokine of the invention and at least one other therapeutically active compound.

In some embodiments, the pharmaceutical composition is used for the treatment of an oncological disease.

In some embodiments, the oncological disease is selected from the group comprising: HNSCC (head and neck squamous cell carcinoma), cervical cancer, cancer of unknown primary, glioblastoma, esophageal cancer, bladder cancer, TNBC (triple-negative breast cancer), CRC (colorectal cancer), hepatocellular carcinoma, melanoma, NSCLC (non-small cell lung cancer), kidney cancer, ovarian cancer, colorectal cancer with microsatellite instability, leukemia (acute leukemia or myeloblastic leukemia), lymphoma, multiple myeloma, breast cancer, prostate cancer, bladder cancer, sarcoma, hepatocellular carcinoma, glioblastoma, Hodgkin's lymphoma, T- and B-cell acute lymphoblastic leukemia, small cell lung cancer, refractory non-Hodgkin's B-cell lymphoma, follicular lymphoma, marginal zone B-cell lymphoma, diffuse large B-cell lymphoma, pancreatic cancer, ovarian cancer, higher-risk myelodysplastic syndrome.

In some embodiments of the pharmaceutical composition, other therapeutically active compound is used that is an antibody, chemotherapeutic agent, or hormone therapy agent.

In some embodiments of the pharmaceutical composition, other therapeutically active compound is used that is an immune checkpoint inhibitor.

In some embodiments of the pharmaceutical composition, an immune checkpoint inhibitor is used that is selected from a PD-1 inhibitor, PD-L1 inhibitor, or CTLA-4 inhibitor.

In some embodiments of the pharmaceutical composition, a PD-L1 inhibitor is used that is an antibody that specifically binds to PD-L1.

In some embodiments of the pharmaceutical composition, an antibody is used that specifically binds to PD-L1 and is selected from the group comprising durvalumab, avelumab, atezolizumab, manelimab.

In some embodiments of the pharmaceutical composition, a PD-1 inhibitor is used that is an antibody that specifically binds to PD-1.

In some embodiments of the pharmaceutical composition, an antibody is used that specifically binds to PD-1 and is selected from the group comprising prolgolimab, pembrolizumab, nivolumab.

In some embodiments of the pharmaceutical composition, a CTLA-4 inhibitor is used that is an antibody that specifically binds to CTLA-4.

In some embodiments of the pharmaceutical composition, an antibody is used that specifically binds to CTLA-4 and is ipilimumab.

In one aspect, the present invention relates to a pharmaceutical combination for activating the human IL-10Rα receptor, which comprises the above immunocytokine of the invention and at least one other therapeutically active compound.

In some embodiments, the pharmaceutical combination is used for the treatment of an oncological disease.

In some embodiments, the oncological disease is selected from the group comprising: HNSCC (head and neck squamous cell carcinoma), cervical cancer, cancer of unknown primary, glioblastoma, esophageal cancer, bladder cancer, TNBC (triple-negative breast cancer), CRC (colorectal cancer), hepatocellular carcinoma, melanoma, NSCLC (non-small cell lung cancer), kidney cancer, ovarian cancer, colorectal cancer with microsatellite instability, leukemia (acute leukemia or myeloblastic leukemia), lymphoma, multiple myeloma, breast cancer, prostate cancer, bladder cancer, sarcoma, hepatocellular carcinoma, glioblastoma, Hodgkin's lymphoma, T- and B-cell acute lymphoblastic leukemia, small cell lung cancer, refractory non-Hodgkin's B-cell lymphoma, follicular lymphoma, marginal zone B-cell lymphoma, diffuse large B-cell lymphoma, pancreatic cancer, ovarian cancer, higher-risk myelodysplastic syndrome.

In some embodiments of the pharmaceutical combination, other therapeutically active compound is used that is an antibody, chemotherapeutic agent, or hormone therapy agent.

In some embodiments of the pharmaceutical combination, other therapeutically active compound is used that is an immune checkpoint inhibitor.

In some embodiments of the pharmaceutical combination, an immune checkpoint inhibitor is used that is selected from a PD-1 inhibitor, PD-L1 inhibitor, or CTLA-4 inhibitor.

In some embodiments of the pharmaceutical combination, a PD-L1 inhibitor is used that is an antibody that specifically binds to PD-L1.

In some embodiments of the pharmaceutical combination, an antibody is used that specifically binds to PD-L1 and is selected from the group comprising durvalumab, avelumab, atezolizumab, manelimab.

In some embodiments of the pharmaceutical combination, a PD-1 inhibitor is used that is an antibody that specifically binds to PD-1.

In some embodiments of the pharmaceutical combination, an antibody is used that specifically binds to PD-1 and is selected from the group comprising prolgolimab, pembrolizumab, nivolumab.

In some embodiments of the pharmaceutical combination, a CTLA-4 inhibitor is used that is an antibody that specifically binds to CTLA-4.

In some embodiments of the pharmaceutical combination, an antibody is used that specifically binds to CTLA-4 and is ipilimumab.

In one aspect, the present invention relates to a method for treatment of an oncological disease, which comprises administering to a subject in need of such treatment the above immunocytokine of the invention or any above pharmaceutical composition, in a therapeutically effective amount.

In some embodiments of the method for treatment, the oncological disease is selected from the group comprising: HNSCC (head and neck squamous cell carcinoma), cervical cancer, cancer of unknown primary, glioblastoma, esophageal cancer, bladder cancer, TNBC (triple-negative breast cancer), CRC (colorectal cancer), hepatocellular carcinoma, melanoma, NSCLC (non-small cell lung cancer), kidney cancer, ovarian cancer, colorectal cancer with microsatellite instability, leukemia (acute leukemia or myeloblastic leukemia), lymphoma, multiple myeloma, breast cancer, prostate cancer, bladder cancer, sarcoma, hepatocellular carcinoma, glioblastoma, Hodgkin's lymphoma, T- and B-cell acute lymphoblastic leukemia, small cell lung cancer, refractory non-Hodgkin's B-cell lymphoma, follicular lymphoma, marginal zone B-cell lymphoma, diffuse large B-cell lymphoma, pancreatic cancer, ovarian cancer, higher-risk myelodysplastic syndrome.

In one aspect, the present invention relates to a method for activating the human IL-10Rα receptor in a subject in need of such activation, which comprises administering to a subject in need of such treatment an effective amount of the above immunocytokine of the invention or any above pharmaceutical composition, in a therapeutically effective amount.

In one aspect, the present invention relates to the use of the above immunocytokine of the invention or any above pharmaceutical composition for the treatment of an oncological disease in a subject in need of such treatment.

In one aspect, the present invention relates to the use of the above immunocytokine of the invention or at least one other therapeutically active compound for the treatment in a subject in need of such treatment of an oncological disease.

In some embodiments of the use, the oncological disease is selected from the group comprising: HNSCC (head and neck squamous cell carcinoma), cervical cancer, cancer of unknown primary, glioblastoma, esophageal cancer, bladder cancer, TNBC (triple-negative breast cancer), CRC (colorectal cancer), hepatocellular carcinoma, melanoma, NSCLC (non-small cell lung cancer), kidney cancer, ovarian cancer, colorectal cancer with microsatellite instability, leukemia (acute leukemia or myeloblastic leukemia), lymphoma, multiple myeloma, breast cancer, prostate cancer, bladder cancer, sarcoma, hepatocellular carcinoma, glioblastoma, Hodgkin's lymphoma, T- and B-cell acute lymphoblastic leukemia, small cell lung cancer, refractory non-Hodgkin's B-cell lymphoma, follicular lymphoma, marginal zone B-cell lymphoma, diffuse large B-cell lymphoma, pancreatic cancer, ovarian cancer, higher-risk myelodysplastic syndrome.

In some embodiments of the use, other therapeutically active compound is used that is an antibody, chemotherapeutic agent, or hormone therapy agent.

In some embodiments of the use, other therapeutically active compound is used that is an immune checkpoint inhibitor.

In some embodiments of the use, an immune checkpoint inhibitor is used that is selected from a PD-1 inhibitor, PD-L1 inhibitor, or CTLA-4 inhibitor.

In some embodiments of the use, a PD-L1 inhibitor is used that is an antibody that specifically binds to PD-L1.

In some embodiments of the use, an antibody is used that specifically binds to PD-L1 and is selected from the group comprising durvalumab, avelumab, atezolizumab, manelimab.

In some embodiments of the use, a PD-1 inhibitor is used that is an antibody that specifically binds to PD-1.

In some embodiments of the use, an antibody is used that specifically binds to PD-1 and is selected from the group comprising prolgolimab, pembrolizumab, nivolumab.

In some embodiments of the use, a CTLA-4 inhibitor is used that is an antibody that specifically binds to CTLA-4.

In some embodiments of the use, an antibody is used that specifically binds to CTLA-4 and is ipilimumab.

### BRIEF DESCRIPTION OF DRAWINGS

FIG. 1 is a map of the plasmid pIntA-IL-10Ra-exc-hum-EPEA comprising a sequence of the extracellular portion of the human IL-10R receptor subunit alpha with an EPEA tag linked to C terminal.
AmpR is a gene that provides resistance to ampicillin
pUC is a bacterial origin of replication
cmv-enhancer is a cytomegalovirus enhancer
hCMV promoter is a human cytomegalovirus eukaryotic promoter
Intron A is a sequence of intron A
Kozak is a Kozak consensus sequence for translation initiation
mIgK Leader is a leader peptide
SalI is a SalI restriction site
IL-10Ra-exc-hum is the extracellular portion of the human IL-10 receptor subunit alpha
EPEA-tag is an EPEA tag sequence for protein purification
KpnI is a KpnI restriction site
Stop is a stop codon
SV40_PA term is a poly(A) SV40 transcription terminator
EBV ori is an eukaryotic origin of replication

FIG. 2 is a map of the plasmid pIntA-N-Fc-LALA-link-hIL-10 comprising a sequence of the N-terminal human IgG1 Fc fragment having mutations LALA, del GK and cross-linked to human IL-10 via a linker.
AmpR is a gene that provides resistance to ampicillin
pUC is a bacterial origin of replication
cmv-enhancer is a cytomegalovirus enhancer
hCMV promoter is a human cytomegalovirus eukaryotic promoter
Intron A is a sequence of intron A
Kozak is a Kozak consensus sequence for translation initiation
mIgK Leader is a leader peptide
SalI is a SalI restriction site
Fc-LALA-DELGK is a sequence of the human IgG1 Fc fragment with a LALA mutation (L19A, L20A according to sequential numbering) and deleted GK (G231, K232 according to sequential numbering) at the C-terminal end
Linker is a linker sequence
hIL-10 is a human IL-10 sequence
XbaI is an XbaI restriction site
Stop is a stop codon
SV40_PA term is a poly(A) SV40 transcription terminator
EBV ori is an eukaryotic origin of replication

FIG. 3 is PAAG electrophoresis of the extracellular portion of human, cynomolgus and rabbit IL-10 receptors derived and isolated from CHO cells.
1) Protein molecular weight markers
2) Human excIL-10Ra
3) Cynomolgus excIL-10Ra
4) Protein molecular weight markers
5) Rabbit excIL-10Ra

FIG. 4 is PAAG electrophoresis of the recombinant immunocytokine (fusion protein) based on IL-10 and human IgG1 Fc fragment of the invention (IL-10-Fc) derived and isolated from CHO cells.
1) Protein molecular weight markers
2) IL10-Fc 4.5 μg under reducing conditions
3) IL10-Fc 4.5 μg under non-reducing conditions.

FIG. 5 is a graph showing the stability of the immunocytokine (fusion protein) based on IL-10 and human IgG1 Fc fragment of the invention (IL-10+Fc) when stored in human serum for a long time at +37C. X-axis is the storage time, Y-axis is the relative content of stable molecules in serum (percent of the original sample).

FIG. 6 is a graph showing the ability of the immunocytokine (fusion protein) based on IL-10 and human IgG1 Fc fragment of the invention (IL-10+Fc) to exhibit proliferative activity as compared to that of IL-10. X-axis is the concentration of IL-10 (positive control human recombinant IL-10 from Peprotech), Y-axis is the relative fluorescence reflecting the number of viable cells. The half maximal effective concentration for the control sample was 91.7 pM, that for IL-10+Fc of the invention was 53.3 pM.

FIG. 8 is a graph showing the effect of the immunocytokine (fusion protein) based on IL-10 and human IgG1 Fc fragment of the invention (IL-10+Fc) on the cytotoxicity of CD8+T cells against Raji target cells as compared to that of recombinant human IL-10 from Peprotech and control.

FIG. 9 is a graph showing the absence of CDC activity in the test immunocytokine (fusion protein) based on IL-10 and human IgG1 Fc fragment of the invention (IL-10+Fc) in an assay employing the Jurkat cell line. The control antibody Rituximab induces complement-mediated lysis of target cells.

FIG. 10 is a graph showing the absence of antibody-dependent cellular cytotoxicity (ADCC activity) in the test immunocytokine (fusion protein) based on IL-10 and human IgG1 Fc fragment of the invention (IL-10+Fc) in an assay employing the Jurkat target cells. Anti-PD1 antibody was used as a positive control.

FIG. 11 is a histogram showing the absence of autocytotoxicity in the immunocytokine (fusion protein) based on IL-10 and human IgG1 Fc fragment of the invention (IL-10+Fc) with respect to subpopulations of human peripheral blood cells at concentrations of 0.01 μg/ml, 0.1 μg/ml, 1 μg/ml, 10 μg/ml:

FIG. 11A) CD4+T cells,

FIG. 11B) CD8+T cells,

FIG. 11C) T cells,

FIG. 11D) NK cells,

FIG. 11E) B cells.

Anti-CD20 antibody inducing autocytotoxicity against B cells and anti-CD47 antibody inducing autocytotoxicity against NK cells were used as a positive control.

FIG. 12 is a histogram reflecting the ability of the immunocytokine (fusion protein) based on IL-10 and human IgG1 Fc fragment of the invention (IL-10-Fc) and a combination of IL-10-Fc of the invention with anti-mPD1 antibody to exert antitumor activity in Balb/c mice vaccinated with CT26 carcinoma. Anti-MPD1 antibody was used as a control.

FIG. 13 is a histogram reflecting the ability of the immunocytokine (fusion protein) based on IL-10 and human IgG1 Fc fragment of the invention (IL-10-Fc) and a combination of IL-10-Fc of the invention with anti-mPD1 antibody to exert antitumor activity in Balb/c mice vaccinated with CT26 carcinoma. Anti-MPD1 antibody was used as a control. Y-axis is the index of tumor growth (ITG).

FIG. 14 is a histogram reflecting the ability of the immunocytokine (fusion protein) based on IL-10 and human IgG1 Fc fragment of the invention (IL-10-Fc) and a combination of IL-10-Fc of the invention with anti-mPD1 antibody to exert antitumor activity in Balb/c mice vaccinated with CT26 carcinoma; the histogram shows the indicator of tumor growth inhibition (TGI). Anti-MPD1 antibody was used as a control.

FIG. 15 is a schematic representation of the format of the immunocytokine (fusion protein) based on IL-10 and human IgG1 Fc fragment of the invention

### DESCRIPTION OF THE INVENTION

Definitions and General Methods

Unless defined otherwise herein, all technical and scientific terms used in connection with the present invention will have the same meaning as is commonly understood by those skilled in the art.

Further, unless otherwise required by context, singular terms shall include plural terms, and the plural terms shall include the singular terms. Typically, the present classification and methods of cell culture, molecular biology, immunology, microbiology, genetics, analytical chemistry, organic synthesis chemistry, medical and pharmaceutical chemistry, as well as hybridization and chemistry of protein and nucleic acids described herein are well known by those skilled and widely used in the art. Enzyme reactions and purification methods are performed according to the manufacturer's guidelines, as is common in the art, or as described herein.

“Binding affinity” generally refers to the strength of the sum total of noncovalent interactions between a single binding site of a molecule (e.g. an antibody) and its binding partner (e.g. an antigen). Unless indicated otherwise, “binding affinity” refers to intrinsic (characteristic, true) binding affinity which reflects a 1:1 interaction between members of a binding pair (e.g. antibody and antigen). The affinity of a molecule X for its binding partner Y can generally be represented by the dissociation constant (Kd). The preferred Kd value is about 200 nM, 150 nM, 100 nM, 60 nM, 50 nM, 40 nM, 30 nM, 20 nM, 10 nM, 8 nM, 6 nM, 4 nM, 2 nM, 1 nM, or less. Affinity can be measured by common methods known in the art, including those described in the present description. Low-affinity antibodies generally bind an antigen slowly and tend to dissociate readily, whereas high-affinity antibodies generally bind an antigen faster and tend to remain bound longer. A variety of methods of measuring binding affinity are known in the art, any of which can be used for the purposes of the present invention.

The term “in vitro” refers to a biological entity, a biological process, or a biological reaction outside the body under artificial conditions. For example, a cell grown in vitro is to be understood as a cell grown in an environment outside the body, e.g. in a test tube, a culture vial, or a microtiter plate.

The term “IC50” (inhibitory concentration 50%), as used herein, refers to concentrations of a formulation, at which a measurable activity or response, for example, growth/proliferation of cells such as tumor cells, is inhibited by 50%. IC50 value can be calculated using appropriate dose-response curves, using special statistical software for curve fitting.

The term “ED50” (EC50) (50% effective dose/concentration) refers to concentrations of a formulation producing 50% biological effect (which may include cytoxicity).

As used in the present description and claims that follow, unless otherwise dictated by the context, the words “have”, “include,” and “comprise” or variations thereof such as “has”, “having,” “includes”, “including”, “comprises,” or “comprising,” will be understood to imply the inclusion of a stated integer or group of integers but not the exclusion of any other integer or group of integers.

Immunocytokine

As surprisingly found by the authors, the immunocytokine comprising a homodimeric complex based on IL-10 and human IgG1 Fc fragment provided in the present invention has an extended plasma half-life as compared to that of IL-10 and is stable due to resistance to serum protease degradation thanks to the unique structure thereof.

The format of the above immunocytokine is shown in FIG. 15.

The term “immunocytokine” means a molecule comprising an antibody or fragments thereof directly or indirectly linked by covalent bonds to a cytokine or derivatives thereof. Said antibody and said cytokine can be linked by a linker peptide.

The immunocytokine of the invention is contemplated to refer to an isolated immunocytokine.

The immunocytokine of the invention refers to a fusion protein based on IL-10 and human IgG1 Fc fragment.

The term “isolated” used to describe various immunocytokines of this description means an immunocytokine which has been identified and separated and/or regenerated from a cell or cell culture, in which the immunocytokine is expressed. Impurities (contaminant components) from natural environment are materials which typically interfere with diagnostic or therapeutic uses of the polypeptide, and may include enzymes, hormones, and other proteinaceous or nonproteinaceous solutes. The isolated polypeptide is typically prepared by at least one purification step.

The immunocytokine of the invention is a recombinant immunocytokine.

The term “recombinant immunocytokine” is intended to refer to an immunocytokine that is expressed in a cell or cell line comprising nucleotide sequence(s) encoding the immunocytokine, wherein said nucleotide sequence(s) is not naturally associated with the cell.

The fragment crystallizable region (“Fc region, Fc fragment”) of an immunoglobulin is the “tail” region of an immunoglobulin molecule that interacts with cell surface Fc-receptor, as well as some proteins of the complement system. This property allows antibodies to activate the immune system. In IgG, IgA and IgD isotypes, the Fc region is composed of two identical protein fragments, respectively, from the second and third constant domains of the two heavy chains; in IgM and IgE isotypes, the Fc contains three heavy chain constant domains (CH2, CH3, and CH4 domains) in each polypeptide chain.

The receptor for IL-10 is a heterotetramer complex comprising two IL-10Rα (also referred to as IL-10R1) molecules encoded by the IL-10ra gene and two IL10Rβ (also referred to as IL-10R2) molecules encoded by the IL-10rb gene.

The human IL-10Rα receptor (or CD210a, or IL-10R1) refers to interleukin 10 receptor, alpha subunit. The molecular weight of IL-10Rα is 90-120 kDa and serves as a ligand-binding subunit of the receptor complex.

In one aspect, the present invention relates to an immunocytokine for activating the human IL-10Rα receptor, which comprises a homodimeric complex based on IL-10 and a human IgG1 Fc fragment, i.e. a dimer that includes two identical monomers based on IL-10 and a human IgG1 Fc fragment, wherein the monomer comprises the amino acid sequence

(SEQ ID NO: 1)
EPKSSDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGGGSGGGPGSGGSPG
QGTQSENSCTHFPGNLPNMLRDLRDAFSRVKTFFQMKDQLDNLLLKESL
LEDFKGYLGCQALSEMIQFYLEEVMPQAENQDPDIKAHVNSLGENLKTL
RLRLRRCHRFLPCENKSKAVEQVKNAFNKLQEKGIYKAMSEFDIFINYI
EAYMTMKIRN.

In one embodiment, the present invention relates to an immunocytokine for activating the human IL-10Rα receptor, which comprises a homodimeric complex based on IL-10 and a human IgG1 Fc fragment, i.e. a dimer that includes two identical monomers based on IL-10 and a human IgG1 Fc fragment, wherein the monomer has the amino acid sequence of SEQ ID NO:1.

The immunocytokine of the invention includes the L234A and L235A (LALA) mutations in the Fc fragment of a human IgG1 antibody to provide effectorless properties of the immunocytokine. These mutations are present in the above immunocytokine, which includes the amino acid sequence with SEQ ID NO: 1.

In one embodiment, the immunocytokine of the invention comprises a leader peptide.

In one embodiment of the invention, the immunocytokine of the invention with a leader peptide comprises a homodimeric complex based on a leader peptide, IL-10 and a human IgG1 Fc fragment, i.e. a dimer that includes two identical monomers based on a leader peptide, IL-10 and human IgG1 Fc fragment, wherein the monomer has the amino acid sequence

(SEQ ID NO: 2)
MMSFVSLLLVGILFHATQAEPKSSDKTHTCPPCPAPEAAGGPSVFLFPP
KPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSL
SLSPGGGSGGGPGSGGSPGQGTQSENSCTHFPGNLPNMLRDLRDAFSRV
KTFFQMKDQLDNLLLKESLLEDFKGYLGCQALSEMIQFYLEEVMPQAEN
QDPDIKAHVNSLGENLKTLRLRLRRCHRFLPCENKSKAVEQVKNAFNKL
QEKGIYKAMSEFDIFINYIEAYMTMKIRN.

The terms “immunocytokine of the invention”, “immunocytokine based on IL-10 and Fc”, “fusion protein of the invention”, “IL-10-Fc” and “IL-10+Fc” are used interchangeably herein and refer to the above immunocytokine of the invention.

Nucleic Acid

In one aspect, the present invention relates to an isolated nucleic acid that encodes the above immunocytokine of the invention.

The terms “nucleic acid”, “nucleic sequence”, “nucleic acid sequence”, “polynucleotide”, “oligonucleotide”, “polynucleotide sequence” and “nucleotide sequence”, used interchangeably in the present description, mean a precise sequence of nucleotides, modified or not, determining a fragment or a region of a nucleic acid, containing unnatural nucleotides or not, and being either a double-strand DNA or RNA, a single-strand DNA or RNA, or transcription products of said DNAs.

It should also be included here that the present invention does not relate to nucleotide sequences in their natural chromosomal environment, i.e. in a natural state. The sequences of the present invention have been isolated and/or purified, i.e., they were sampled directly or indirectly, for example by copying, their environment having been at least partially modified. Thus, isolated nucleic acids obtained by recombinant genetics, by means, for example, of host cells, or obtained by chemical synthesis should also be mentioned here.

An “isolated” nucleic acid molecule is one which is identified and separated from at least one nucleic acid molecule-impurity, which the former is bound to in the natural source. An isolated nucleic acid molecule is different from the form or set in which it is found under natural conditions. Thus, an isolated nucleic acid molecule is different from a nucleic acid molecule that exists in cells under natural conditions. An isolated nucleic acid molecule however includes a nucleic acid molecule located in cells in which the immunocytokine is normally expressed, for example, if the nucleic acid molecule has a chromosomal localization that is different from its localization in cells under natural conditions.

In some embodiments, the isolated nucleic acid is DNA.

In one embodiment, the present invention relates to a nucleic acid molecule comprising a nucleotide sequence that encodes a monomer of a fusion protein based on IL-10 and a human IgG1 Fc fragment, wherein the monomer comprises the amino acid sequence of SEQ ID NO: 1.

In one embodiment, the present invention relates to a nucleic acid molecule comprising a nucleotide sequence that encodes a monomer of a fusion protein based on IL-10 and a human IgG1 Fc fragment, wherein the monomer has the amino acid sequence of SEQ ID NO: 1.

As would be appreciated by those skilled in the art, because of the redundancy of the genetic code, a variety of different DNA sequences can encode the fusion protein with the amino acid sequence of SEQ ID NO: 1. It is well within the skill of a person trained in the art to create these alternative DNA sequences encoding the same amino acid sequences. Such variant DNA sequences are within the scope of the present invention.

A reference to a nucleotide sequence encompasses the complement thereof unless otherwise specified. Thus, a reference to a nucleic acid having a particular sequence should be understood as one which encompasses the complementary strand thereof with the complementary sequence thereof.

In some embodiments, the isolated nucleic acid that encodes the monomer of the fusion protein based on IL-10 and a human IgG1 Fc fragment, wherein the monomer comprises the amino acid sequence of SEQ ID NO: 1, comprises the nucleotide sequence with SEQ ID NO: 3.

In some embodiments, the isolated nucleic acid that encodes the monomer of the fusion protein based on IL-10 and human IgG1 Fc fragment, wherein the monomer has the amino acid sequence of SEQ ID NO: 1, has the nucleotide sequence with SEQ ID NO: 3.

In some embodiments, the isolated nucleic acid that encodes the monomer of the fusion protein based on IL-10 and a human IgG1 Fc fragment, wherein the monomer comprises the amino acid sequence of SEQ ID NO: 1, comprises the nucleotide sequence with SEQ ID NO: 4.

In some embodiments, the isolated nucleic acid that encodes the monomer of the fusion protein based on IL-10 and human IgG1 Fc fragment, wherein the monomer has the amino acid sequence of SEQ ID NO: 1, has the nucleotide sequence with SEQ ID NO: 4.

In some embodiments, the nucleic acid of the invention encodes the monomer of the fusion protein based on a leader peptide, IL-10, and human IgG1 Fc fragment.

In some embodiments, the nucleic acid that encodes the monomer of the fusion protein based on a leader peptide, IL-10, and human IgGl Fc fragment, wherein the monomer has the amino acid sequence of SEQ ID NO: 2, has the nucleotide sequence with SEQ ID NO: 5.

In some embodiments, the nucleic acid that encodes the monomer of the fusion protein based on a leader peptide, IL-10, and human IgGl Fc fragment, wherein the monomer has the amino acid sequence of SEQ ID NO: 2, has the nucleotide sequence with SEQ ID NO: 6.

The above nucleic acid molecules may be used to express the immunocytokine of the invention.

Expression Vector

In one aspect, the present invention relates to an expression vector comprising any of the above nucleotide sequences.

The present invention relates to a vector suitable for the expression of any of nucleotide sequences described herein.

The term “vector” as used herein means a nucleic acid molecule capable of transporting another nucleic acid to which it has been linked. In some embodiments, a vector is a plasmid, i.e., a circular double stranded piece of DNA into which additional DNA segments may be ligated. In some embodiments, a vector is a viral vector, wherein additional DNA segments may be ligated into the viral genome.

In some embodiments, vectors are capable of autonomous replication in a host cell into which they are introduced (e.g. bacterial vectors having a bacterial origin site of replication and episomal mammalian vectors). In further embodiments, vectors (e.g. non-episomal mammalian vectors) may be integrated into the genome of a host cell upon introduction into a host cell, and thereby are replicated along with the host gene. Moreover, certain vectors are capable of directing the expression of genes to which they are operably linked. Such vectors are referred to herein as “recombinant expression vectors” (or simply, “expression vectors”).

The present invention relates to vectors comprising nucleic acid molecules that encode the amino acid sequence of the immunocytokine (fusion protein) of the invention, portions thereof (e.g., IL-10 sequences or sequences of the second and third constant domains of the Fc fragment), as described herein. The invention further relates to vectors comprising nucleic acid molecules encoding the immunocytokine (fusion protein) based on IL-10 and human IgG1 Fc fragment of the invention or fragments thereof.

Expression vectors include plasmids, retroviruses, adenoviruses, adeno-associated viruses (AAVs), plant viruses, such as cauliflower mosaic virus, tobacco mosaic virus, cosmids, YACs, EBV derived episomes, and the like. DNA molecules may be ligated into a vector such that transcriptional and translational control sequences within the vector serve their intended function of regulating the transcription and translation of the DNA. An expression vector and expression control sequences may be chosen to be compatible with the expression host cell used. DNA molecules can be introduced into an expression vector by standard methods (e.g. ligation of complementary restriction sites on an immunocytokine gene fragment and vector, or blunt end ligation if no restriction sites are present).

The term “control sequences” refers to DNA sequences necessary for the expression of an operably linked coding sequence in a particular host organism. The control sequences that are suitable for prokaryotes are, for example, a promoter, optionally an operator sequence and a ribosome binding site. Eukaryotic cells are known to include promoters, polyadenylation signals, and enhancers.

Nucleic acid is “operably linked” when it is placed into a functional relationship with another nucleic acid sequence. For example, DNA for a presequence or secretory leader sequence is operably linked to DNA for a polypeptide if it is expressed as a preprotein that participates in the secretion of the polypeptide; a promoter or enhancer is operably linked to a coding sequence if it affects the transcription of the sequence; a ribosome binding site is operably linked to a coding sequence if it is positioned so as to facilitate translation. Typically, “operably linked” means that the DNA sequences being linked are contiguous, and, in the case of a secretory leader sequence, are contiguous and in reading phase. However, enhancers do not have to be contiguous.

A recombinant expression vector can also encode a signal (leader) peptide that facilitates the secretion of the immunocytokine from a host cell. The immunocytokine gene may be cloned into a vector such that the signal (leader) peptide is linked in-frame to the amino terminus of the immunocytokine chain. The signal (leader) peptide can be an immunoglobulin signal (leader) peptide or a heterologous signal peptide (i.e., a signal (leader) peptide from a non-immunoglobulin protein).

The recombinant vector expression of the invention can carry regulatory sequences that control the expression of immunocytokine genes in a host cell. It will be understood by those skilled in the art that the design of an expression vector, including the selection of regulatory sequences, may depend on such factors as the choice of a host cell to be transformed, the level of expression of a desired protein, and so forth. Preferred control sequences for an expression host cell in mammals include viral elements that ensure high levels of protein expression in mammalian cells, such as promoters and/or enhancers derived from a retroviral LTR, cytomegalovirus (CMV) (such as a CMV promoter/enhancer), simian virus 40 (SV40) (such as a SV40 promoter/enhancer), adenovirus, (e.g. the major late promoter adenovirus (AdMLP)), polyomavirus and strong mammalian promoters such as native immunoglobulin promoter or actin promoter.

Methods for expressing polypeptides in bacterial cells or fungal cells, e.g. yeast cells, are also well known in the art.

In addition to the immunocytokine genes and regulatory sequences, the recombinant expression vectors of the invention may carry additional sequences, such as sequences that regulate replication of a vector in host cells (e.g. origins of replication) and selectable marker genes. The selectable marker gene facilitates the selection of host cells into which a vector has been introduced.

The term “expression control sequence” as used in the present description refers to polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include the promoter of ribosome binding site, and transcription termination sequences; in eukaryotes, typically, such control sequences include promoters and transcription termination sequences. The term “control sequences” includes at least all components, the presence of which is essential for expression and processing, and can also include additional components, the presence of which is advantageous, for example, leader sequences and fusion partner sequences.

Host Cells and Method for Production Thereof

In one aspect, the present invention relates to a method for production of a host cell for production of the immunocytokine of the invention, which comprises transformation of the cell with the above vector.

In one aspect, the present invention relates to a host cell for production of the immunocytokine of the invention, the host cell comprises any of the above nucleic acids.

The term “recombinant host cell” (or simply “host cell”) as used herein refers to a cell into which a recombinant expression vector has been introduced. The present invention relates to host cells, which may include, for example, a vector according to the invention described above. The present invention further relates to host cells that include any of the nucleic acids encoding the immunocytokine of the present invention. It should be understood that “recombinant host cell” and “host cell” refer not only to a particular subject cell but to the progeny of such a cell as well. Since modifications may occur in succeeding generations due to either mutation or environmental influences, such progeny may not, in fact, be identical to a parental cell; however, such cells are still included within the scope of the term “host cell” as used herein.

Nucleic acid molecules encoding immunocytokines of the invention and vectors comprising these nucleic acid molecules can be used for transfection of a suitable mammalian or cell thereof, plant or cell thereof, bacterial or yeast host cell. Transformation can be carried out by any known technique of introducing polynucleotides into a host cell. Methods for introduction of heterologous polynucleotides into mammalian cells are well known in the art and include dextran--mediated transfection, cationic polymer-nucleic acid complex transfection, calcium phosphate precipitation, polybrene-mediated transfection, protoplast fusion, encapsulation of the polynucleotide(s) in liposomes, and direct microinjection of DNA into nuclei. In addition, nucleic acid molecules may be introduced into mammalian cells by viral vectors.

Mammalian cell lines used as hosts for transformation are well known in the art and include a plurality of immortalized cell lines available. These include, e.g., Chinese hamster ovary (CHO) cells, NS0 cells, SP2 cells, HEK-293T cells, FreeStyle 293 cells (Invitrogen), NIH-3T3 cells, HeLa cells, baby hamster kidney (BHK) cells, African green monkey kidney cells (COS), human hepatocellular carcinoma cells (e.g., Hep G2), A549 cells, and a number of other cell lines. Cell lines are selected by determining which cell lines have high expression levels and provide for necessary characteristics of the protein produced. Other cell lines that may be used are insect cell lines, such as Sf9 or Sf21 cells. When the recombinant expression vectors encoding the immunocytokine of the invention are introduced into mammalian host cells, the immunocytokine is produced by culturing the host cells for a period of time sufficient to express the immunocytokine of the invention in the host cells, or, more preferably, secrete the immunocytokine into the culture medium in which the host cells are cultured. The immunocytokine of the invention can be isolated from culture medium using standard protein purification techniques. Plant host cells include, e.g. Nicotiana, Arabidopsis, duckweed, corn, wheat, potato, etc. Bacterial host cells include Escherichia and Streptomyces species. Yeast host cells include Schizosaccharomyces pombe, Saccharomyces cerevisiae and Pichia pastoris.

Furthermore, level of production of the immunocytokine of the invention from a producing cell line can be enhanced using a number of known techniques. For example, the glutamine synthetase gene expression system (the GS system) is a common approach for enhancing expression under certain conditions.

It is likely that the immunocytokine of the invention in different cell lines or transgenic animals will have different glycosylation patterns from each other. However, the immunocytokine of the invention encoded by nucleic acid molecules described herein, or comprising amino acid sequences provided herein are part of the present invention, regardless of the glycosylation of the binding molecules, and, in general, regardless of the presence or absence of post-translational modifications.

The Above Host Cell Does Not Refer to a Host Cell Produced Using Human Embryos.

The above host cell does not refer to a host cell produced by modifying the genetic integrity of human germline cells.

In one aspect, the present invention relates to a method for preparing a product comprising the immunocytokine of the invention, which comprises culturing of the above host cell in a culture medium under conditions sufficient to produce said immunocytokine, if necessary, followed by isolation and purification of the resulting immunocytokine.

The present invention relates to methods for producing the immunocytokine of the invention. One embodiment of the invention relates to a method for producing the immunocytokine as defined herein, comprising preparing a recombinant host cell capable of expressing the immunocytokine of the invention, culturing said host cell under conditions suitable for expression/production of the immunocytokine of the invention, and isolating the resulting immunocytokine. An immunocytokine produced by such expression in such recombinant host cells is referred to herein as a “recombinant immunocytokine”. The invention also relates to the progeny of cells from such host cells and the immunocytokine produced analogously.

Pharmaceutical Composition and Pharmaceutical Combination

In one aspect, the present invention relates to a pharmaceutical composition for activating the human IL-10Rα receptor, which comprises the immunocytokine of the invention and one or more pharmaceutically acceptable excipients.

“Pharmaceutical composition” refers to a composition comprising an immunocytokine of the invention and at least one of components selected from the group comprising pharmaceutically acceptable and pharmacologically compatible fillers, solvents, diluents, carriers, auxiliary, distributing and sensing agents, delivery agents, such as preservatives, stabilizers, filler, disintegrators, moisteners, emulsifiers, suspending agents, thickeners, sweeteners, flavouring agents, aromatizing agents, antibacterial agents, fungicides, lubricants, and prolonged delivery controllers, the choice and suitable proportions of which depend on the type and way of administration and dosage. Examples of suitable suspending agents are ethoxylated isostearyl alcohol, polyoxyethene, sorbitol and sorbitol ether, microcrystalline cellulose, aluminum metahydroxide, bentonite, agar-agar and tragacant and their mixtures as well. Protection against action of microorganisms can be provided by various antibacterial and antifungal agents, such as, for example, parabens, chlorobutanole, sorbic acid, and similar compounds. The composition may also contain isotonic agents, such as, for example, sugars, polyols, sodium chloride, and the like. Prolonged action of the composition may be achieved by agents slowing down absorption of active ingredient, for example, aluminum monostearate and gelatine. Examples of suitable carriers, solvents, diluents and delivery agents include water, ethanol, polyalcohols and their mixtures, natural oils (such as olive oil) and organic esters (such as ethyl oleate) for injections. Examples of fillers are lactose, milk sugar, sodium citrate, calcium carbonate, calcium phosphate, and the like. Examples of disintegrators and distributors are starch, alginic acid and its salts, silicates and the like. Examples of suitable lubricants are magnesium stearate, sodium lauryl sulfate, talc and polyethylene glycol of high molecular weight. The pharmaceutical composition for peroral, sublingual, transdermal, intraocular, intramuscular, intravenous, subcutaneous, local or rectal administration of active ingredient, alone or in combination with another active compound may be administered to human and animals in a standard administration form, in a mixture with traditional pharmaceutical carriers. Suitable standard administration forms include peroral forms such as tablets, gelatin capsules, pills, powders, granules, chewing-gums and peroral solutions or suspensions; sublingual and transbuccal administration forms; aerosols; implants; local, transdermal, subcutaneous, intramuscular, intravenous, intranasal or intraocular forms and rectal administration forms.

The term “excipient” is used herein to describe any ingredient other than the immunocytokine of the invention. These are substances of inorganic or organic nature which are used in the pharmaceutical manufacturing in order to give drug products the necessary physicochemical properties.

The term “pharmaceutically acceptable” refers to one or more compatible liquid or solid components that are suitable for administration in a mammal, preferably a human.

The term “buffer”, “buffer composition”, “buffering agent” refers to a solution, which is capable of resisting changes in pH by the action of its acid-base conjugate components, and which allows the immunocytokine product to resist changes in pH. Generally, the pharmaceutical composition preferably has a pH in the range from 4.0 to 8.0. Examples of buffers used include, but are not limited to, acetate, phosphate, citrate, histidine, succinate, etc. buffer solutions.

The terms “tonic agent”, “osmolyte” or “osmotic agent”, as used herein, refer to an excipient that can adjust the osmotic pressure of a liquid formulation of the immunocytokine of the invention. “Isotonic” drug is a drug that has an osmotic pressure equivalent to that of human blood. Isotonic drugs typically have an osmotic pressure from about 250 to 350 mOsm/kg. Isotonic agents used include, but are not limited to, polyols, saccharides and sucrose, amino acids, metal salts, for example, sodium chloride, etc.

“Stabilizer” refers to an excipient or a mixture of two or more excipients that provide the physical and/or chemical stability of the active agent. Stabilizers include amino acids, for example, but are not limited to, arginine, histidine, glycine, lysine, glutamine, proline; surfactants, for example, but are not limited to, polysorbate 20 (trade name: Tween 20), polysorbate 80 (trade name: Tween 80), polyethylene-polypropylene glycol and copolymers thereof (trade names: Poloxamer, Pluronic, sodium dodecyl sulfate (SDS); antioxidants, for example, but are not limited to, methionine, acetylcysteine, ascorbic acid, monothioglycerol, sulfurous acid salts, etc.; chelating agents, for example, but are not limited to, ethylenediaminetetraacetic acid (EDTA), diethylenetriaminepentaacetic acid (DTPA), sodium citrate, etc.

In one aspect, the present invention relates to a pharmaceutical composition for activating the human IL-10Rα receptor, which comprises the immunocytokine of the invention and at least one other therapeutically active compound.

In some embodiments of the pharmaceutical composition, other therapeutically active compound is used that is an antibody, chemotherapeutic agent, or hormone therapy agent.

In some embodiments of the pharmaceutical composition, other therapeutically active compound is used that is an immune checkpoint inhibitor.

In some embodiments of the pharmaceutical composition, an immune checkpoint inhibitor is used that is selected from a PD-1 inhibitor, PD-L1 inhibitor, or CTLA-4 inhibitor.

In some embodiments of the pharmaceutical composition, a PD-L1 inhibitor is used that is an antibody that specifically binds to PD-L1.

In some embodiments of the pharmaceutical composition, an antibody is used that specifically binds to PD-L1 and is selected from the group comprising durvalumab, avelumab, atezolizumab, manelimab.

In some embodiments of the pharmaceutical composition, a PD-1 inhibitor is used that is an antibody that specifically binds to PD-1.

In some embodiments of the pharmaceutical composition, an antibody is used that specifically binds to PD-1 and is selected from the group comprising prolgolimab, pembrolizumab, nivolumab.

In some embodiments of the pharmaceutical composition, a CTLA-4 inhibitor is used that is an antibody that specifically binds to CTLA-4.

In some embodiments of the pharmaceutical composition, an antibody is used that specifically binds to CTLA-4 and is ipilimumab.

In one aspect, the present invention relates to a pharmaceutical combination for activating the human IL-10Rα receptor, which comprises the above immunocytokine of the invention and at least one other therapeutically active compound.

In some embodiments of the pharmaceutical combination, other therapeutically active compound is used that is an antibody, chemotherapeutic agent, or hormone therapy agent.

In some embodiments of the pharmaceutical combination, other therapeutically active compound is used that is an immune checkpoint inhibitor.

In some embodiments of the pharmaceutical combination, an immune checkpoint inhibitor is used that is selected from a PD-1 inhibitor, PD-L1 inhibitor, or CTLA-4 inhibitor.

In some embodiments of the pharmaceutical combination, a PD-L1 inhibitor is used that is an antibody that specifically binds to PD-L1.

In some embodiments of the pharmaceutical combination, an antibody is used that specifically binds to PD-L1 and is selected from the group comprising durvalumab, avelumab, atezolizumab, manelimab.

In some embodiments of the pharmaceutical combination, a PD-1 inhibitor is used that is an antibody that specifically binds to PD-1.

In some embodiments of the pharmaceutical combination, an antibody is used that specifically binds to PD-1 and is selected from the group comprising prolgolimab, pembrolizumab, nivolumab.

In some embodiments of the pharmaceutical combination, a CTLA-4 inhibitor is used that is an antibody that specifically binds to CTLA-4.

In some embodiments of the pharmaceutical combination, an antibody is used that specifically binds to CTLA-4 and is ipilimumab.

In some embodiments, the pharmaceutical composition or pharmaceutical combination is used for the treatment of an oncological disease.

“Treat”, “treating” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment.

The terms “oncological disease”, “cancer” and “cancerous” refer to a physiological condition or describe a physiological condition in mammals that is typically characterized by unregulated growth/proliferation of cells. Examples of oncological diseases include, but are not limited to, carcinoma, lymphoma, blastoma, sarcoma, and leukemia. More particular examples of such cancerous diseases include squamous cell cancer, small-cell lung cancer, non-small cell lung cancer, adenocarcinoma of the lung, squamous carcinoma of the lung, peritoneal cancer, hepatocellular cancer, gastrointestinal cancer including stomach cancer, pancreatic cancer, glioblastoma, glioma, cervical cancer, ovarian cancer, liver cancer, bladder cancer, breast cancer, colon cancer, colorectal cancer, endometrial or uterine carcinoma, salivary gland carcinoma, kidney cancer (renal cell carcinoma), prostate cancer, vulval cancer, thyroid cancer, hepatic carcinoma, anal carcinoma, penile carcinoma, melanoma, and various head and neck cancers.

In one aspect, the subject of treatment, or patient, is a mammal, preferably a human subject. Said subject may be either male or female, of any age.

The immunocytokine of the invention within the pharmaceutical composition or combination is present in a therapeutically effective amount.

“Therapeutically effective amount” refers to that amount of the therapeutic agent being administered during treatment which will relieve to some extent one or more of the symptoms of the disease being treated.

In some embodiments, the oncological disease is selected from the group comprising: HNSCC (head and neck squamous cell carcinoma), cervical cancer, cancer of unknown primary, glioblastoma, esophageal cancer, bladder cancer, TNBC (triple-negative breast cancer), CRC (colorectal cancer), hepatocellular carcinoma, melanoma, NSCLC (non-small cell lung cancer), kidney cancer, ovarian cancer, colorectal cancer with microsatellite instability, leukemia (acute leukemia or myeloblastic leukemia), lymphoma, multiple myeloma, breast cancer, prostate cancer, bladder cancer, sarcoma, hepatocellular carcinoma, glioblastoma, Hodgkin's lymphoma, T- and B-cell acute lymphoblastic leukemia, small cell lung cancer, refractory non-Hodgkin's B-cell lymphoma, follicular lymphoma, marginal zone B-cell lymphoma, diffuse large B-cell lymphoma, pancreatic cancer, ovarian cancer, higher-risk myelodysplastic syndrome.

Pharmaceutical compositions or pharmaceutical combinations of the present invention and methods of preparation thereof will be undoubtedly apparent to those skilled in the art. The pharmaceutical compositions or pharmaceutical combinations should preferably be manufactured in compliance with the GMP (Good Manufacturing Practice) requirements. The composition or combination may comprise a buffer composition, tonicity agents, stabilizers and solubilizers. Prolonged action of the composition or combination may be achieved by agents slowing down absorption of active pharmaceutical ingredient, for example, aluminum monostearate and gelatine. Examples of suitable carriers, solvents, diluents and delivery agents include water, ethanol, polyalcohols and their mixtures, oils, and organic esters for injections.

Any method for administering an immunocytokine accepted in the art may be suitably employed for the immunocytokine of the invention.

The pharmaceutical composition or pharmaceutical combination is “stable” if the active agent retains physical stability and/or chemical stability and/or biological activity thereof during the specified shelf life at storage temperature, for example, of 2-8° C. Preferably, the active agent retains both physical and chemical stability, as well as biological activity. Storage period is adjusted based on the results of stability test in accelerated or natural aging conditions.

A pharmaceutical composition or pharmaceutical combination of the invention can be manufactured, packaged, or widely sold in the form of a single unit dose or a plurality of single unit doses in the form of a ready formulation. The term “single unit dose” as used herein refers to discrete quantity of a pharmaceutical composition containing a predetermined quantity of an active ingredient. The quantity of the active ingredient typically equals the dose of the active ingredient to be administered in a subject, or a convenient portion of such dose, for example, half or a third of such dose.

Pharmaceutical compositions or pharmaceutical combinations according to the present invention are typically suitable for parenteral administration as sterile formulations intended for administration in a human body through the breach in skin or mucosal barriers, bypassing the gastrointestinal tract by virtue of injection, infusion and implantation. In particular, parenteral administration includes, inter alfa, subcutaneous, intraperitoneal, intramuscular, intravenous, intraarterial, intrathecal, intraventricular, intraurethral, intracranial, intrasynovial, transdermal injection or infusion, and kidney dialytic infusion techniques. Intra-tumor delivery, for example, intra-tumor injection, can also be employed. Regional perfusion is also contemplated.

Preferred embodiments include intravenous and subcutaneous routes. Any method for administering peptides or proteins accepted in the art may be suitably employed for the immunocytokine of the invention.

Injectable formulations may be prepared, packaged, or sold, without limitation, in unit dosage form, such as in ampoules, vials, in plastic containers, pre-filled syringes, autoinjection devices. Formulations for parenteral administration include, inter alia, suspensions, solutions, emulsions in oily or aqueous bases, pastes, and the like.

In another embodiment, the invention provides a medicinal formulation for parenteral administration, wherein the pharmaceutical composition or pharmaceutical combination is provided in dry (i.e. powder or granular) form for reconstitution with a suitable base (e.g. sterile pyrogen-free water) prior to administration. Such medicinal formulation may be prepared by, for example, lyophilization, i.e. a process, which is known in the art as freeze drying, and which involves freezing a product followed by removal of solvent from frozen material.

An immunocytokine of the invention can also be administered intranasally or by inhalation, either alone, as a mixture with a suitable pharmaceutically acceptable excipient from an inhaler, such as a pressurized aerosol container, pump, spray, atomiser, or nebuliser, wherein a suitable propellant is used or not used, or as nasal drops, or spray.

Dosage forms for parenteral administration may be formulated to be immediate or modified release. Modified release medicinal formulations include delayed-, sustained-, pulsed-, controlled-, targeted and programmed release.

Method for Treatment/Use for Treatment

In one aspect, the present invention relates to a method for treatment of an oncological disease, which comprises administering to a subject in need of such treatment the immunocytokine of the invention or any above pharmaceutical composition of the invention, in a therapeutically effective amount.

In one aspect, the present invention relates to the use of the immunocytokine of the invention or any above pharmaceutical composition of the invention for the treatment in a subject in need of such treatment of an oncological disease.

In some embodiments of the method for treatment, the oncological disease is selected from the group comprising: HNSCC (head and neck squamous cell carcinoma), cervical cancer, cancer of unknown primary, glioblastoma, esophageal cancer, bladder cancer, TNBC (triple-negative breast cancer), CRC (colorectal cancer), hepatocellular carcinoma, melanoma, NSCLC (non-small cell lung cancer), kidney cancer, ovarian cancer, colorectal cancer with microsatellite instability, leukemia (acute leukemia or myeloblastic leukemia), lymphoma, multiple myeloma, breast cancer, prostate cancer, bladder cancer, sarcoma, hepatocellular carcinoma, glioblastoma, Hodgkin's lymphoma, T- and B-cell acute lymphoblastic leukemia, small cell lung cancer, refractory non-Hodgkin's B-cell lymphoma, follicular lymphoma, marginal zone B-cell lymphoma, diffuse large B-cell lymphoma, pancreatic cancer, ovarian cancer, higher-risk myelodysplastic syndrome.

In the case of a tumor (for example, cancer), the therapeutically effective amount of an immunocytokine of the invention may reduce the number of cancer cells; reduce the initial tumor size; inhibit (i.e. slow to some extent and preferably stop) cancer cell infiltration into surrounding organs; inhibit (i.e. slow to some extent and preferably stop) tumor metastasis; inhibit to some extent tumor growth; and/or relieve to some extent one or more of the symptoms associated with the disease. The immunocytokine of the invention may to some extent prevent growth and/or kill existing cancer cells, it may be cytostatic and/or cytotoxic. For cancer therapy, in vivo efficacy can, for example, be measured by assessing survival, time to tumor progression (progression-free survival), tumor response rate to treatment (TRR), duration of response and/or quality of life.

The immunocytokine of the invention can be administered without further therapeutic treatment, i.e. as an independent therapy. Furthermore, treatment by the immunocytokine of the invention may comprise at least one additional therapeutic treatment (combination therapy). In some embodiments, the immunocytokine of the invention may be administered jointly or formulated with another medication/preparation for the treatment of cancer.

As used herein, the terms “co-administration”, “co-administered” and “in combination with”, referring to the immunocytokine of the invention and one or more other therapeutic agents, are contemplated to mean, refer to or include the following:
1) simultaneous administration of such combination of an immunocytokine of the invention and a therapeutic agent to a patient in need of treatment, when such components are formulated together into a single dosage form which releases said components at substantially the same time to said patient,
2) simultaneous administration of such combination of an immunocytokine of the invention and a therapeutic agent to a patient in need of treatment, when such components are formulated apart from each other into separate dosage forms which are taken at substantially the same time by said patient, whereupon said components are released at substantially the same time to said patient,
3) sequential administration of such combination of an immunocytokine of the invention and a therapeutic agent to a patient in need of treatment, when such components are formulated apart from each other into separate dosage forms which are taken at consecutive times by said patient with a significant time interval between each administration, whereupon said components are released at substantially different times to said patient; and
4) sequential administration of such combination of an immunocytokine of the invention and a therapeutic agent to a patient in need of treatment, when such components are formulated together into a single dosage form which releases said components in a controlled manner, whereupon they are concurrently, consecutively, and/or overlappingly released at the same and/or different times to said patient, where each part may be administered by either the same or a different route.

In one aspect, the present invention relates to the use of the immunocytokine of the invention or at least one other therapeutically active compound for the treatment in a subject in need of such treatment of an oncological disease.

The immunocytokine of the present invention can be combined with a therapeutic agent selected from the group comprising: a cytotoxic agent, a chemotherapeutic agent, a hormone therapy agent, or another therapeutic antibody.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, including fragments and/or variants thereof.

A “chemotherapeutic agent” is a chemical compound useful in the treatment of a malignant neoplasm. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylmelamine; acetogenins (e.g. bullatacin and bullatacinone); delta-9-tetrahydrocannabinol (dronabinol MARINOL®); beta-lapachone; lapachol; colchicines; betulinic acid; camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (e.g. cryptophycin 1 and cryptophycin 8); dolastatin; duocarmycin (including the synthetic analogues, KW-2189 and CB1-TM1); eleutherobin; pancratistatin; sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g. calicheamicin, e.g. calicheamicin gamma II and calicheamicin omega II (see, e.g. Agnew, Chem. Intl. Ed. Engl., 33: 183-186 (1994)); dynemicin, including dynemicin A; esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXOL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), epothilone, and 5-fluorouracil (5-FU); folic acid analogues such as denopterin, methotrexate, pteropterin,trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (e.g., T-2 toxin, verracurin A, roridin A and anguidine); urethan; dacarbazine; mannomustine; mitobronitol; mitolactol; pipobroman; gacyto sine; arabinoside (“Ara-C”); thiotepa; taxoid, e.g., paclitaxel (TAXOL), albumin-engineered nanoparticle formulation of paclitaxel (ABRAXANE), and docetaxel (TAXOTERE®); chlorambucil; 6-thioguanine; mercaptopurine; methotrexate; platinum agents such as cisplatin, oxaliplatin, and carboplatin; vinca alkaloids, which prevent tubulin polymerization from forming microtubules, including vinblastine (VELBAN®), vincristine (ONCOVIN®), vindesine (ELDISINE®), FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluorometlhylornithine (DIVITO); retinoids such as retinoic acid, including bexarotene (TARGRETIN®); biphosphonates such as clodronate (for example, BONEFOS®or OSTAC®), etidronate (DIDROCAL®), NE-58095, zoledronic acid/zoledronate (ZOMETA®), alendronate (FOSAMAJX®), pamidronate (AREDIA®), tiludronate (SKELID®), or risedronate (ACTONEL®); troxacitabine (1,3-dioxolane nucleoside cytosine analog); antisense oligonucleotides, e.g. those that inhibit expression of genes in signaling pathways implicated in aberrant cell proliferation, such as for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®), BAY439006 (sorafenib; Bayer); SU-11248 (Pfizer); perifosine, COX-2 inhibitor (e.g., celecoxib or etoricoxib), proteosome inhibitor (e.g., PS341); bortezomib (VELCADE®); CCI-779; tipifarnib (811577); orafenib, ABT510; Bcl-2 inhibitor such as oblimersen sodium (GENASENSE®); pixantrone; EGFR inhibitors (see definition below); tyrosine kinase inhibitors (see definition below); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATINTM) combined with 5-FU and leucovovin.

Also included in this definition are hormone therapy agents that act to regulate or inhibit hormone action on tumors, such as anti-estrogens with mixed agonist/antagonist profile, including, tamoxifen (NOLVADEX®), 4-hydroxytamoxifen, trioxifene, toremifene (FARESTON®), idoxifene, droloxifene, raloxifene (EVTSTA®), trioxifene, keoxifene, and selective estrogen receptor modulators (SERMs), such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors, such as formestane and exemestane (AROMASIN®), and nonsteroidal aromatase inhibitors, such as anastrazole (AREVIIDEX®), letrozole (FEMARA®) and aminoglutethimide, and other aromatase inhibitors including vorozole (RIVISOR®), megestrol acetate (MEGASE®), fadrozole, imidazole; lutenizing hormone-releasing hormone agonists, including leuprolide (LUPRON® and ELIGARD®), goserelin, buserelin, and tripterelin; sex steroids, including progestines, such as megestrol acetate and medroxyprogesterone acetate, estrogens, such as diethylstilbestrol and premarin, and androgens/retinoids such as fluoxymesterone, fully transretionic acid and fenretinide; onapristone; anti-progesterones; estrogen receptor down-regulators (ERDs); anti-androgens, such as flutamide, nilutamide and bicalutamide; testolactone; and pharmaceutically acceptable salts, acids or derivatives of any of the above; as well as combinations of two or more of the above.

The other therapeutic agent that can be used in combination with the immunocytokine of the present invention may be a therapeutic antibody selected from the group comprising antibodies to PD-1 (e.g. prolgolimab, pembrolizumab or nivolumab), antibodies to PD-L1, antibodies to CTLA4, antibodies to 4-1BB, antibodies to OX40, antibodies to GITR, antibodies to CD20 (e.g. rituximab), antibodies to HER2 (e.g. trastuzumab or pertuzumab), antibodies to VEGF (e.g. bevacizumab), or combinations thereof.

In some embodiments of the use, other therapeutically active compound is used that is an immune checkpoint inhibitor.

The term “immune checkpoint inhibitor” (or “checkpoint inhibitor”) refers to compounds inhibiting the activity of immune checkpoints. Inhibition includes reduction of function and full blockade. Examples of inhibitory checkpoint molecules include B7-H3, B7-H4, BTLA, CTLA-4, KIR, PD-1, PD-L1, PD-L2, LAG-3, TIM-3, TIGIT, and VISTA. In some embodiments, the immune checkpoint inhibitor is an antibody that specifically recognizes an immune checkpoint protein. A number of immune checkpoint inhibitors are known and in analogy of these known immune checkpoint protein inhibitors, alternative immune checkpoint inhibitors may be developed in the near future. The immune checkpoint inhibitors include, but are not limited to, peptides, antibodies, nucleic acid molecules, and low molecular weight compounds.

In some embodiments of the use, an immune checkpoint inhibitor is used that is selected from a PD-1 inhibitor, PD-L1 inhibitor, or CTLA-4 inhibitor.

In some embodiments of the use, a PD-L1 inhibitor is used that is an antibody that specifically binds to PD-L1.

In some embodiments of the use, an antibody is used that specifically binds to PD-L1 and is selected from the group comprising durvalumab, avelumab, atezolizumab, manelimab.

In some embodiments of the use, a PD-1 inhibitor is used that is an antibody that specifically binds to PD-1.

In some embodiments of the use, an antibody is used that specifically binds to PD-1 and is selected from the group comprising prolgolimab, pembrolizumab, nivolumab.

In some embodiments of the use, a CTLA-4 inhibitor is used that is an antibody that specifically binds to CTLA-4.

In some embodiments of the use, an antibody is used that specifically binds to CTLA-4 and is ipilimumab.

In one aspect, the present invention relates to a method for activating the human IL-10Rα receptor in a subject in need of such activation, which comprises administering to a subject in need of such treatment an effective amount of the immunocytokine of the invention or any above pharmaceutical composition of the invention, in a therapeutically effective amount.

It is understood that an immunocytokine of the invention may be used in methods for treating, as described above, in the use for treatment, as described above, and/or in the manufacture of a medicament for the therapeutic applications described above.

Doses and Routes of Administration

The immunocytokine of the present invention will be administered in an amount that is effective in treatment of the condition in question, i.e. in doses and during the periods of time required to achieve the desired result. A therapeutically effective amount may vary according to factors such as the specific condition to be treated, age, sex, and weight of a patient, and whether the immunocytokine is administered alone or in combination with one or more additional drugs or treatment techniques.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in a unit dosage form for ease of administration and uniformity of dosage. A unit dosage form as used herein is intended to refer to physically discrete units suited as unitary dosages for patients/subjects to be treated; each unit contains a predetermined quantity of active compound calculated to produce the desired therapeutic effect in association with the desired pharmaceutical carrier. Specification for the unit dosage forms of the invention is typically dictated by and directly dependent on (a) the unique characteristics of a chemotherapeutic agent and particular therapeutic or prophylactic effect to be achieved, and (b) the limitations inherent in the art of compounding such an active compound for the treatment of sensitivity in the subjects.

Thus, a skilled artisan would appreciate, based upon the disclosure provided herein, that the doses and dosage regimen are adjusted in accordance with methods well-known in the therapeutic arts. That is, the maximum tolerable dose can be readily established, and the effective amount providing a detectable therapeutic effect to a patient may also be determined, as can the temporal requirements for administering each agent to provide a detectable therapeutic effect to a patient. Thus, while certain dose and administration regimens are exemplified herein, these examples in no way limit the doses and administration regimen that may be provided to a patient in practicing the embodiments of the invention.

It is to be noted that dosage values may vary with the type and severity of the condition to be alleviated, and may include single or multiple doses. Furthermore, it is to be understood that for any particular subject, specific dosage regimens should be adjusted over time according to the individual need and the judgment of a medical professional administering or supervising the administration of the compositions, and that dosage ranges set forth in the present description are exemplary only and are not intended to limit the scope or practice of the claimed compositions. Further, the dosage regimen with the compositions of the present invention may be based on a variety of factors, including the type of disease, the age, weight, sex, medical condition of the patient, the severity of the condition, the route of administration, and the particular immunocytokine of the invention. Thus, the dosage regimen can vary widely, but can be determined routinely using standard methods. For example, doses may be adjusted based on pharmacokinetic and pharmacodynamic parameters, which may include clinical effects such as toxic effects or laboratory values. Thus, the present invention encompasses intra-patient dose-escalation as determined by the person skilled in the art. Methods for determining appropriate dosages and regimens are well-known in the art and would be understood by a skilled artisan once provided the ideas disclosed herein.

Examples of Suitable Administration Methods are Provided Above.

It is contemplated that a suitable dose of an immunocytokine of the invention will be in the range of 0.007-200 mg/kg, preferably 0.007-100 mg/kg, including about 0.5-50 mg/kg, for example about 1-20 mg/kg. The immunocytokine of the present invention may be administered, e.g. in a dose of at least 0.25 mg/kg, such as at least 0.5 mg/kg, including at least 1 mg/kg, e.g. at least 1.5 mg/kg, such as at least 2 mg/kg, e.g. at least 3 mg/kg, including at least 4 mg/kg, e.g. at least 5 mg/kg; and for example up to a maximum of 50 mg/kg, including up to a maximum of 30 mg/kg, e.g. up to a maximum of 20 mg/kg, including up to a maximum of 15 mg/kg. The administration will typically be repeated in appropriate time intervals, such as once a week, once every two weeks, once every three weeks or once every four weeks, and for as long as deemed appropriate by a responsible physician, who may, in some cases, increase or reduce the dose if necessary.

Implementation of the Invention

The following examples are provided for better understanding of the invention. These examples are for purposes of illustration only and are not to be construed as limiting the scope of the invention in any manner.

All publications, patents, and patent applications cited in this specification are incorporated herein by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended embodiments.

Materials and General Methods

General information regarding the nucleotide sequences of human immunoglobulin light and heavy chains is given in: Kabat, E. A., et al., Sequences of Proteins of Immunological Interest, 5th ed., Public Health Service, National Institutes of Health, Bethesda, MD (1991). Amino acids of antibody chains are numbered according to EU numbering (Edelman, G. M., et al., Proc. Natl. Acad. Sci. USA 63 (1969) 78-85; Kabat, E. A. , et al., Sequences of Proteins of Immunological Interest, 5th ed., Public Health Service, National Institutes of Health, Bethesda, MD, (1991).

Recombinant DNA Techniques

Standard methods were used to manipulate DNA as described in Sambrook, J. et al, Molecular cloning: A laboratory manual; Cold Spring Harbor Laboratory Press, Cold Spring Harbor, New York, 1989. The molecular biological reagents were used according to the manufacturer protocols.

Gene Synthesis

Desired gene segments were prepared from oligonucleotides made by chemical synthesis. The gene segments of 300-4000 kb long, which were flanked by singular restriction sites, were assembled by annealing and ligation of oligonucleotides including PCR amplification and subsequently cloned via the indicated restriction sites. The DNA sequences of the subcloned gene fragments were confirmed by DNA sequencing.

DNA Sequence Determination

DNA sequences were determined by Sanger sequencing.

DNA and protein sequence analysis and sequence data management

The Infomax's Vector NT1 Advance suite version 8.0 was used for sequence creation, mapping, analysis, annotation and illustration.

Expression Vectors

For the expression of the described antibodies and antigens, variants of expression plasmids intended for expression in prokaryotic cells (E.coli), transient expression in eukaryotic cells (e.g. in CHO cells) were applied. Beside the antibody expression cassette the vectors comprised: an origin of replication which allows replication of said plasmid in E. coli, genes which confer resistance in E. coli to various antibiotics (e.g. to ampicillin and kanamycin).

The fusion genes comprising the described antibody chains as described below were generated by PCR and/or gene synthesis and assembled with known recombinant methods and techniques by connection of the according nucleic acid segments, e.g. using unique restriction sites in the corresponding vectors. The subcloned nucleic acid sequences were verified by DNA sequencing. For transient transfections, larger quantities of the plasmids were prepared by plasmid preparation from transformed E. coli cultures.

Example 1

Production of sequences of human IL-10Ra genes, and sequences of orthologous IL-10Ra genes of Macaca cynomolgus (fascicularis) and IL-10Ra genes of Oryctolagus cuniculus (Rabbit).

To clone sequences of extracellular portions of the human IL-10Ra receptors (https://www.uniprot.org/uniprot/Q13651) ϰIL-10Ra Macaca cynomolgus (https://www.uniprot.org/uniprot/A0A2K5WJ66), we synthesized oligonucleotides of 60 nucleotides each, forming a completely overlapping gene sequence. Each gene was assembled using two-round PCR, which resulted in the production of a fragment of 642 bp. Next, the extracellular portion of the receptor was cloned into a vector for transient expression of EPEA-tagged pIntA for affinity purification of the protein.

To clone the sequence of the extracellular portion of the Oryctolagus cuniculus IL-10Ra receptor, total RNA was isolated from rabbit peripheral blood cells and cDNA was generated. Reverse transcription products were used as a template in the polymerase chain reaction to produce a receptor gene flanked by restriction sites. Next, the receptor gene was cloned into a vector for transient expression of EPEA-tagged pIntA (FIG. 1) for affinity purification of the protein. The cloned nucleic acid sequences were verified by DNA sequencing.

Example 2

Production of sequence of human IL-10/Fc fragment fusion protein, as well as rabbit IL-10/Fc fragment fusion protein.

To clone a gene comprising a human/rabbit IL-10 sequence and a human IgG1 Fc fragment, genes were fused by PCR and/or gene synthesis and assembly using known recombination methods and protocols by way of connecting the appropriate nucleic acid segments, for example, using SOE-PCR (Splicing by overlap extension). Next, the fusion protein gene was cloned into a vector for transient expression of pIntA (FIG. 2). The cloned nucleic acid sequences were verified by DNA sequencing.

Example 3

Isolation and purification of human, monkey and rabbit IL-10R receptors from mammalian cell suspension culture.

Recombinant proteins (rabbit, human and monkey IL-10Rα receptors, rabbit IL-10 receptor) were generated in the CHO-C cell culture medium. Following transfection of cells with expression vectors, orbital feed-batch cultivation was performed in serum-free medium for 5-10 days. After culturing, cell culture was centrifuged under 2000 g for 20 min and filtered through 0.22 μm filter. Target proteins were isolated from the culture liquid using affinity chromatography on the Akta Pure 25 chromatographic system using an)CK 16 column (GE Healthcare) with 5 ml CaptureSelect C-tag Affinity Matrix (Thermo Scientific) and Superdex 200 Increase 10/300 GL (GE Healthcare). The culture liquid was applied to the column with CaptureSelect C-tag Affinity Matrix, following which the column was washed with 20 mM TrisHCl pH 7 supplemented with 150 mM NaCl and the protein was eluted with 2M MgCl2 solution in 20 mM TrisHCl pH 7 supplemented with 150 mM NaCl. Next, the protein was dialyzed into 20 mM TrisHCl pH 7 with 150 mM NaCl, following which the product was concentrated to 1 ml and applied to a GE Superdex 200 Increase 10/300 GL column equilibrated with a 20 mM TrisHCl pH 7 solution supplemented with 150 mM NaCl. The target peak was collected and the resulting solution was filtered (0.22 μm). The products were stored at −70° C. The purity of the resulting protein solution was evaluated by SDS gel electrophoresis (FIG. 3).

### Example 4

Isolation and purification of immunocytokine (fusion protein) based on IL-10 and human IgG1 Fc fragment (IL-10-Fc) from mammalian cell suspension culture.

The recombinant IL-10-Fc protein of the invention was generated in the CHO-C cell culture medium. Following transfection of cells with expression vectors, orbital feed-batch cultivation was performed in serum-free medium for 5-10 days. The secretion of the immunocytokine in question was monitored using the Pall ForteBio's Octet RED96 system for molecular interactions analysis on protein A biosensors.

After culturing, cell culture was centrifuged under 2000 g for 20 min and filtered through 0.22 μm filter. Target proteins were isolated from the culture liquid by affinity chromatography on the Akta Pure 25 chromatography system using HiTrap rProtein A FF, 5 ml and HiPrep 16/60 Sephacryl S-300 HR columns (GE Healthcare). The culture liquid was applied to the HiTrap rProtein A FF column, following which the column was washed with PBS and the protein was eluted with a solution of 100 mM citrate buffer pH 3, following which the protein solution was neutralized by adding 2M acetate buffer pH 6 at a ratio of 1/10 v/V. Next, the protein was dialyzed into 20 mM acetate buffer pH 6, following which the product was concentrated and applied to a HiPrep 16/60 Sephacryl S-300 HR column equilibrated with a solution of 200 mM acetate buffer pH 6. The target peak was collected and the resulting solution was filtered (0.22 μm). The product was stored at −70° C. The purity of the resulting protein solution was evaluated by SDS gel electrophoresis (FIG. 4).

Example 5

Kinetic studies of affinity and specificity of the interaction between immunocytokine (fusion protein) based on IL-10 and human IgG1 Fc fragment (IL-10-Fc) and human, cynomolgus, murine and rabbit IL-10Rα receptors using Forte Bio OctetRed96.

Binding of human IL-10-Fc of the invention to the human IL-10 receptor, as well as to the cynomolgus, murine and rabbit IL-10Rα receptors, was assessed by bio-layer interferometry using the OctetRed96 device (Pall). IL-10-Fc was bound to AR2G sensors according to the manufacturer's instructions for the preparation and immobilization of AR2G sensors. The sensors with immobilized IL-10 were then immersed into wells containing the human, macaque, murine or rabbit IL-10Rα receptor. Following the association of interleukin and the receptor, the sensors were immersed in the working solution for the subsequent dissociation stage. The resulting sensograms, after subtracting a reference signal, were analyzed using Octet Data Analysis software (Version 8.0) in accordance with the standard procedure and using 1:1 interaction model. According to the data obtained (Table 1), IL-10-Fc of the invention binds to the human, cynomolgus, murine IL-10 receptors.

TABLE 1
Affinity constants for interaction of IL-10-Fc of the invention
with human, cynomolgus, murine and rabbit IL-10 receptors.

| | Affinity constant (KD), M | | | |
|---|---|---|---|---|
| | Human | Cynomolgus | Murine | Rabbit |
| | IL-10 | IL-10 | IL-10 | IL-10 |
| | receptor, M | receptor, M | receptor, M | receptor, M |
| IL- | 5.1 ± 0.3E−09 | 3.7 ± 0.4E−09 | 2.1 ± 0.2E−09 | 2.03E−08 |
| 10-Fc | | | | |

Example 6

Analysis of thermostability of immunocytokine (fusion protein) based on IL-10 and human IgG1 Fc fragment (IL-10-Fc).

The recombinant IL10-Fc protein of the invention in 20 mM acetate buffer, pH 6, was heated using an amplifier in a plastic test tube at 50C for 48 hours, followed by a shift to +4C. After the end of the program, the samples were analyzed before and after heating using analytical gel chromatography on a TSK Gel G3000 SW×1 column. The areas of the target peaks of the samples before and after heating were compared. A 7% change in the area of the dimer peak after heating for 48 hours indicates the stability of the product and the possibility of long-term storage (Table 2).

TABLE 2
Comparison of the ratio of the area of peaks in the chromatograms
of the IL-10-Fc product of the invention before and after heating

| | % ratio of peaks | | |
|---|---|---|---|
| Sample | Σaggregates, % | dimer, % | Σfragments, % |
| Before | 1.80 | 97.25 | 0.96 |
| heating | | | |
| Following | 7.99 | 89.97 | 2.05 |
| heating | | | |

Example 7

Analysis of stability of the immunocytokine (fusion protein) based on IL-10 and human IgG1 Fc fragment (IL-10-Fc) during long-term storage in human serum at 37C.

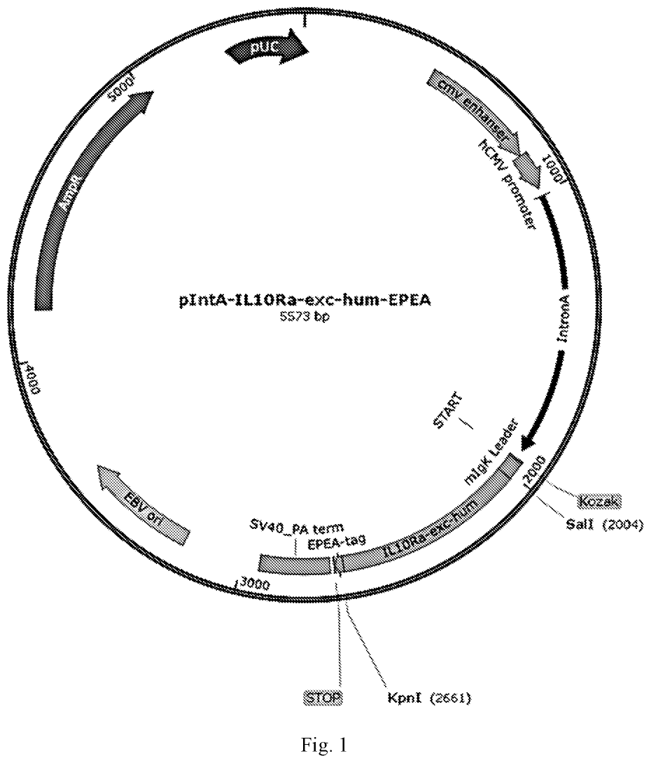

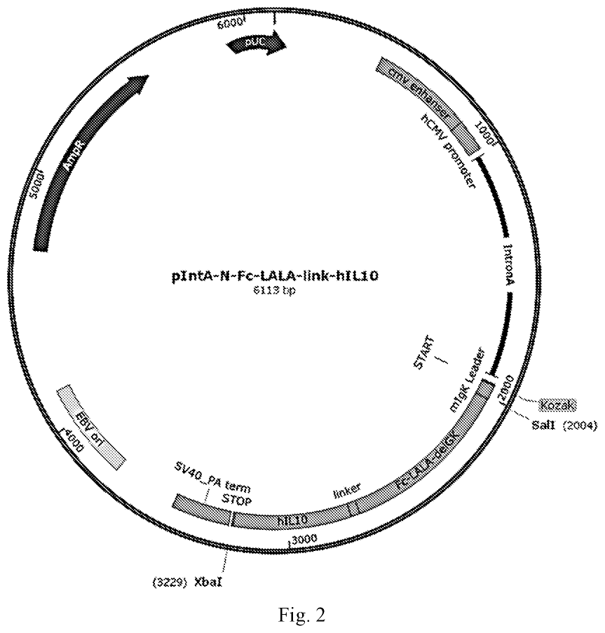

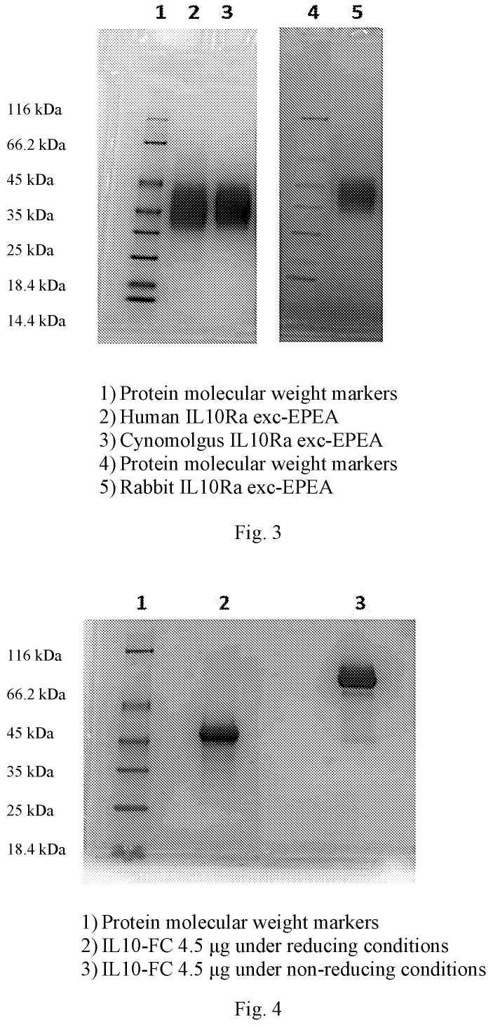

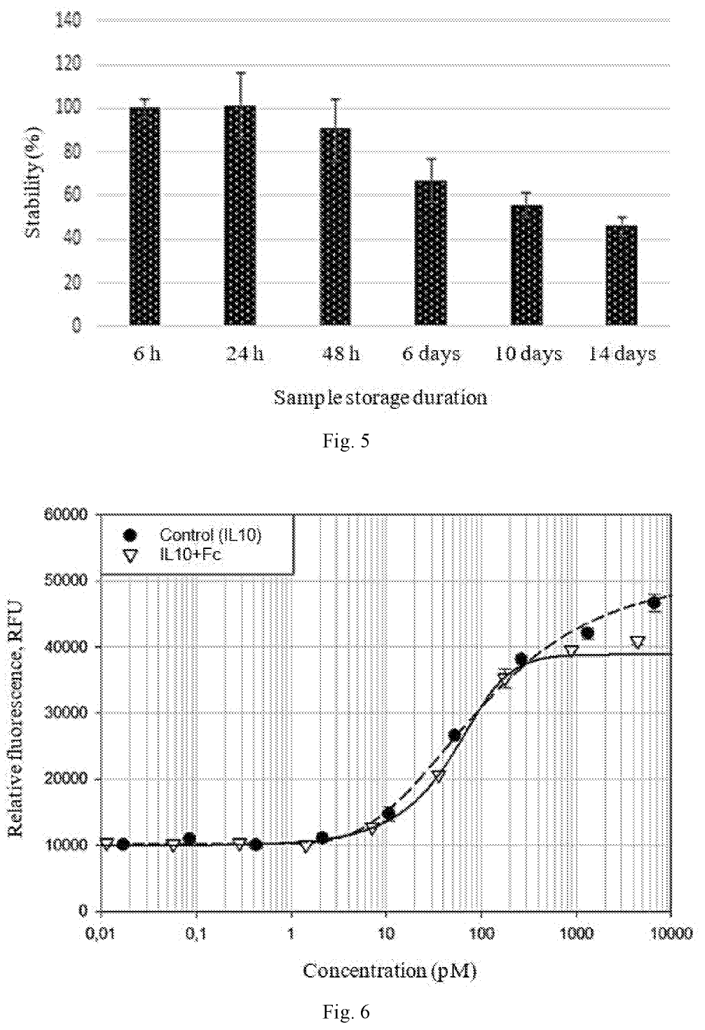

IL-10 has a relatively short half-life in the organism. For example, the half-life in mice as measured by in vitro bioassay or by efficacy in a septic shock simulation system [see Smith et al., Cellular Immunology 173:207-214 (1996)], is about 2 to 6 hours. In the present study, stability was understood as a decrease in the concentration of full-length IL-10 candidate molecules following in vitro storage in human serum at +37° C. To determine the stability, the IL-10-Fc immunocytokine was diluted in human serum to a concentration of 5 μg/ml and stored at +37° C. in a temperature-regulated chamber for 24 h to 14 days. Samples of unsupplemented human serum used for subsequent determination of the background concentration values of the samples in question were stored under similar conditions. After storage, the concentration of the samples was determined by enzyme immunoassay. Data calculation and plotting were performed using the MS Excel software package (FIG. 5).

Thus, IL-10+Fc of the invention has high stability in serum due to resistance to serum protease degradation. The resistance is provided by the structural feature of the given fusion protein, the structure of which being shown in SEQ ID NO: 1.

Example 8

Determination of proliferative activity of immunocytokine (fusion protein) based on IL-10 and human IgG1 Fc fragment (IL-10-Fc) in a cellular assay on MC/9 mouse mast cells.

A critical step for the cytokine function is the interaction of the cytokine with its receptor. Activation of the IL-10Rα receptor by the test candidates was analyzed on the MC/9 cell line in a standard proliferation assay in the presence of the costimulatory cytokine IL4. It has been shown in the literature that IL-10 stimulates the proliferation of MC/9 mouse mast cells in the presence of IL4 (Thompson-Snipes L et al J Exp Med. 1991 doi:10.1084/jem.173.2.507 https://www.ncbi.nlm.nih.gov/pmc/articles/PMC2118779/). The assay is used as a standard assay for assessing the biological activity of commercially available formulations of recombinant human IL-10. The activity of the control product (recombinant human IL-10) is≤2.0 ng/ml in a proliferation assay on the MC/9 line, as stated by the manufacturer (Peprotech).

The assay was carried out in a 96-well culture plate for suspension cell cultures. The suspension contained per well 10,000 MC/9 cells, and the test candidates (recombinant human IL-10, Peprotech catalog number: 200-10, and IL-10+Fc of the invention) at a concentration as indicated in the graph. The final suspension volume was 150 μl per well. All suspension components were prepared in RPMI-1640 medium supplemented with 2 mM glutamine, 10 μg/ml gentamicin, 10% inactivated fetal bovine serum (FBS), and 10 pg/ml IL4. After adding all components, the plate was incubated for 72 h at 37° C. in a humid atmosphere in the presence of 5% CO₂. At the end of the incubation period, 17 μL of Alamar Blue dye was added to the wells and the plate was incubated for 3-6 h at 37° C. in a humid atmosphere in the presence of 5% CO₂ until pink staining was seen. Fluorescence was measured using a plate reader at a wavelength of 495/590 nm.

Thus, IL-10-Fc of the invention activates the IL-10Rα receptor, as confirmed in a proliferation cellular assay on MC/9 cells (FIG. 6). The half maximal effective concentration (EC50) of the test IL-10+Fc immunocytokine of the invention was 53.3 pM in the cellular assay, whereas that of the control recombinant human IL-10 from Peprotech was 91.7 pM. The data obtained indicate that the activity of IL-10+Fc of the invention is higher as compared to that of the commercially available IL-10.

Example 9

Determination of functional activity of immunocytokine (fusion protein) based on IL-10 and human IgG1 Fc fragment (IL-10-Fc) by secretion of IFN-γ by human CD8+T cells.

Antigen-independent activation of TCR and CD28 on CD8+T cells in vitro (via anti-CD3 and anti-CD28 antibodies) causes PD1 expression on the cell surface, which leads to suppression of the activity of cytotoxic lymphocytes followed by depletion thereof. Concurrently, the IL-10Rα receptor is expressed on the cell surface, the activation of which, when interacting with IL-10, has an antiapoptotic effect, which contributes to the survival of cytotoxic T cells producing various cytokines, including IFN-γ, in the tumor microenvironment.

Human peripheral blood mononuclear cells (PBMCs) were isolated from whole blood from a healthy donor by Ficoll density gradient centrifugation. CD8+T cells were then isolated from the suspension of PBMCs on a magnetic column using a commercial kit.

The test was performed in a 96-well plate with anti-CD3 antibodies (1 μg/ml) pre-immobilized in the wells. The suspension contained per well 30,000 CD8+T cells, as well as an anti-CD28 antibody at a concentration of 1 μg/ml. The final volume of the cell suspension and antibodies in a well was 200 μl, all components of the suspension were prepared in RPMI-1640 medium comprising 10% FBS. The plate was incubated for 3 days at 37° C. with 5% CO₂. On day 4 of incubation, the cells were collected and washed from the anti-CD28 antibody by centrifugation. The resulting cell precipitate was suspended in RPMI 1640 medium supplemented with 10% FBS at a concentration of 0.3*10⁶ cells/ml. 100 μl of washed cells and 100 μl of candidate antibodies were introduced at a concentration as specified in the graph to the wells of a new pre-prepared ELISA 96-well plate with anti-CD3 antibody immobilized onto the plastic (1 μg/ml). The plate was incubated for 3 days at 37° C. with 5% CO₂. On day 4 of incubation, aliquots of culture liquid were collected from the wells and the concentration of IFN-γ was measured.

Thus, IL-10-Fc of the invention shows the ability to induce the production of the cytokine IFN-γ by human activated cytotoxic CD8+T cells.

Example 10

Determination of the effect of immunocytokine (fused protein) based on IL-10 and human IgG1 Fc fragment (IL-10-Fc) on the cytotoxicity of human CD8+T cells against Raji target cells.

Human peripheral blood mononuclear cells (PBMCs) were isolated from whole blood from a healthy donor by Ficoll density gradient centrifugation. CD8+T cells were then isolated from the suspension of PBMCs on a magnetic column.

200 μl of a suspension containing 30,000 CD8+T cells and 1 μg/ml of anti-CD28 antibody was introduced to a 96-well plate with anti-CD3 antibodies (1 μg/ml) pre-immobilized in the wells, and incubated for 3 days at 37° C., 5% CO₂. The cells were then collected, washed by centrifugation, suspended in RPMI-1640 medium supplemented with 10% FBS, and injected at 30,000 cells/well into the wells of a new pre-prepared ELISA 96-well plate with anti-CD3 antibody immobilized onto the plastic (1 μg/ml) and candidate antibody solutions at a concentration as specified in the graph. The plate was incubated for 3 days at 37° C. with 5% CO₂. The cells were then collected from the plate wells, washed by centrifugation, and the precipitates were suspended in RPMI-1640 10% medium at a concentration of 0.3*10⁶ cells/ml.

6.5 ml of Calcein AM solution was added to 2*10⁶ Raji cells. Cells with calcein were incubated for 30 minutes at 37° C. in a CO₂ incubator. At the end of incubation, the cells were washed twice from calcein by centrifugation. A suspension with a concentration of 0.3*106 cells/ml was prepared.

The assay was conducted in a 96-well culture plate. The suspension contained per well 30,000 CD8+T cells, 30,000 Raji cells labeled with calcein AM, and an anti-CD3/CD20 antibody at a final concentration of 50 ng/ml. The plate was incubated for 2.5 hours in an incubator at 37° C., 5% CO₂.

30 minutes prior to the end of incubation, a lysis buffer solution was added to the wells under control of maximum lysis. Fluorescence was measured on a plate reader at 485/538 nm.

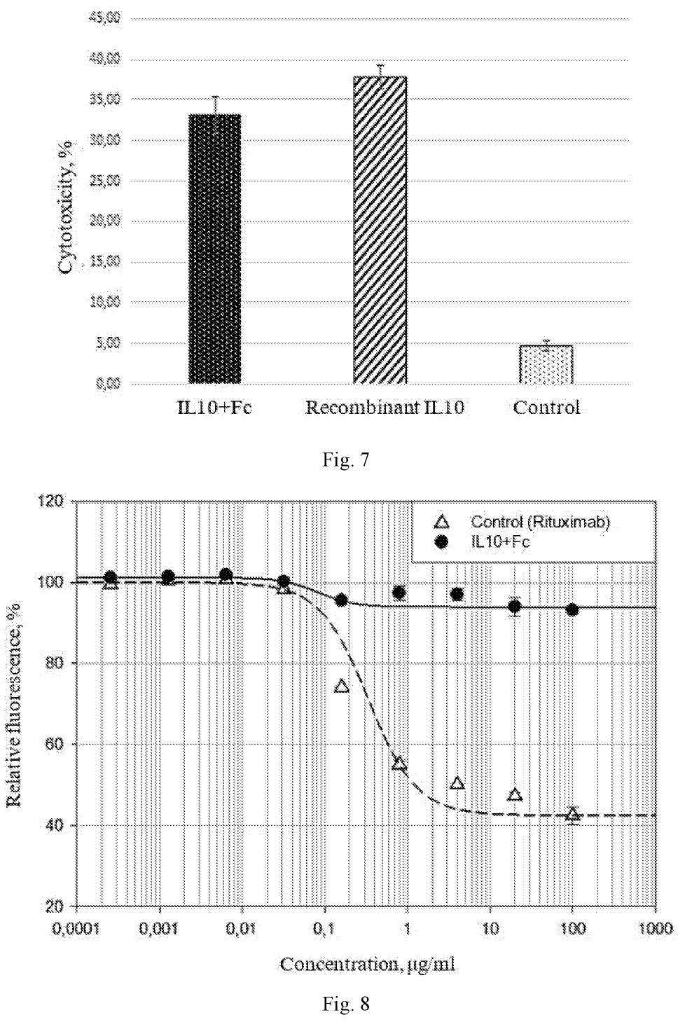

It was shown that culturing of CD8+T cells in the presence of IL-10+Fc of the invention increases their cytotoxic activity against Raji target cells by 7 times as compared to that of the control (FIG. 8).

Example 11

Measurement of CDC activity.

The CDC assay used the Jurkat cell line. The assay was conducted in a 96-well culture plate. The suspension contained per well 50,000 Jurkat cells, as well as IL-10+Fc of the invention at the specified concentration and a human serum complement diluted to 1:4. The final volume of the cell suspension in a well was 150 μl, all components of the suspension were prepared in RPMI-1640 medium comprising 0.1% BSA. The plate was then incubated for 4 h at 37° C. with 5% CO₂. 15 μl of alamar blue reagent was added to each well and incubated at 37° C., 5% CO₂ for 16 hours. An antibody with CDC activity Rituximab (CO83031118R) produced by JSC “Biocad” and Raji target cells, at the same concentration as Jurkat cells, were used as a positive control. Fluorescence was measured at an excitation wavelength of 544 nm and emission wavelength of 590 nm using a plate reader.

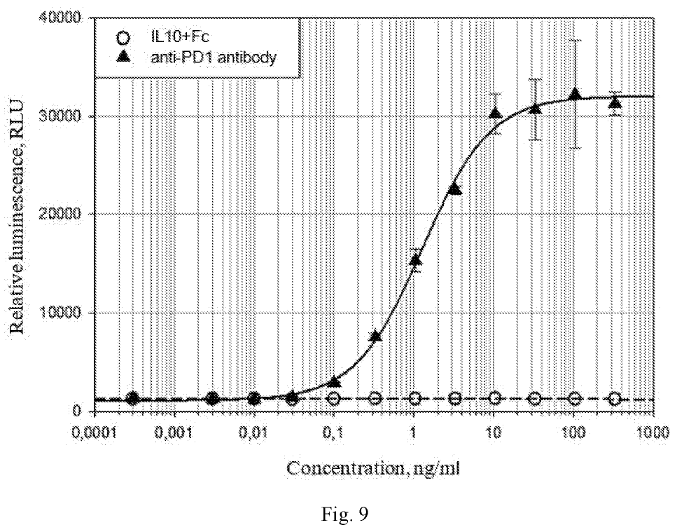

It has been shown that IL-10+Fc of the invention does not induce complement-mediated lysis (CDC) of the Jurkat cell line (FIG. 9).

Example 12

Measurement of ADCC activity.

The assay used a reporter cell line Jurkat-NFAT-Luc-CD16 created on the basis of the Jurkat cell line, stably expressing CD16 on the surface and containing a gene encoding firefly luciferase, under the control of the NFAT promoter; Jurkat-PD1 clone 43 was used as target cells. The assay was performed to confirm the absence of effector properties in the test immunocytokine IL-10+Fc of the invention.

The assay was performed in a white 96-well culture plate designed for luminescence assays. The suspension contained per well 25,000 Jurkat-NFAT-Luc-CD16 effector cells, 25,000 Jurkat-PD1 target cells, and IL-10-Fc of the invention at a concentration as specified in the graph. An effector anti-PD1 antibody produced by JSC “Biocad” was used as a positive control. The final volume of the cell suspension and immunocytokine (IL-10+Fc of the invention) in a well was 75 μl, all components of the suspension were prepared in RPMI-1640 medium comprising 10% FBS. After adding all the components, the plates were incubated for 5 hours at 37° C., 5% CO₂, and then, using a One-Glo luciferase assay kit (Promega), we measured the luminescence intensity in the wells. Luminescence was measured using a plate reader.

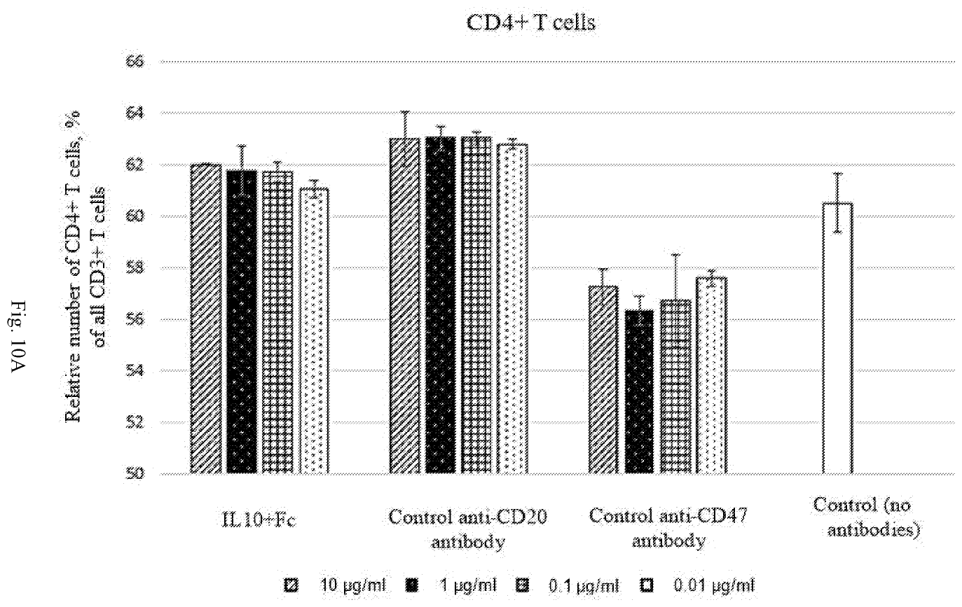

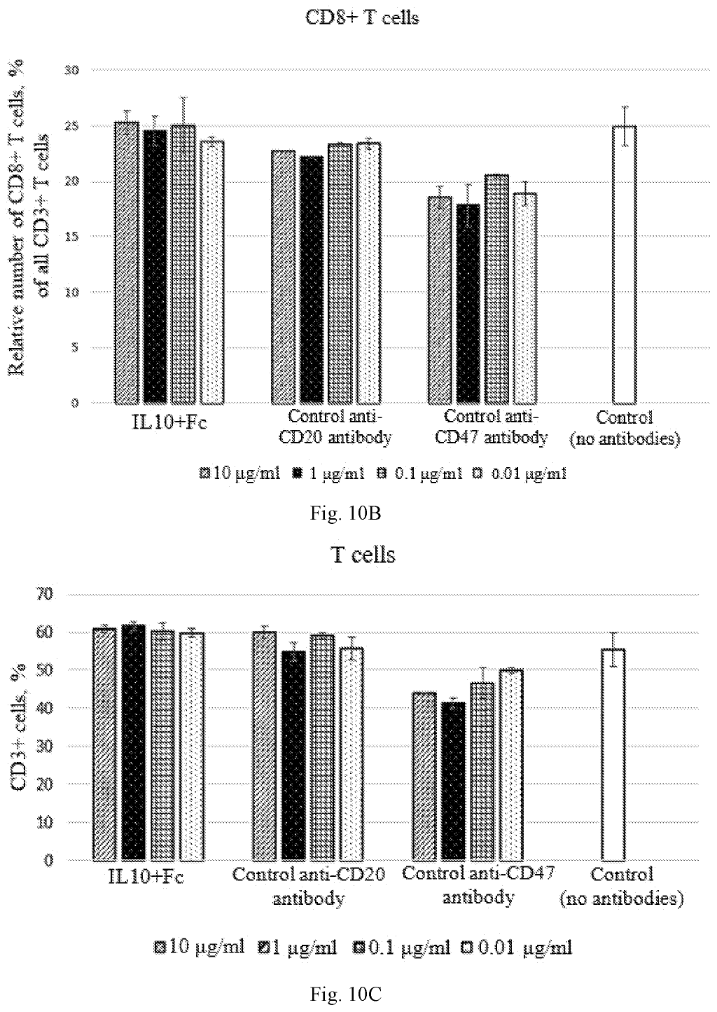

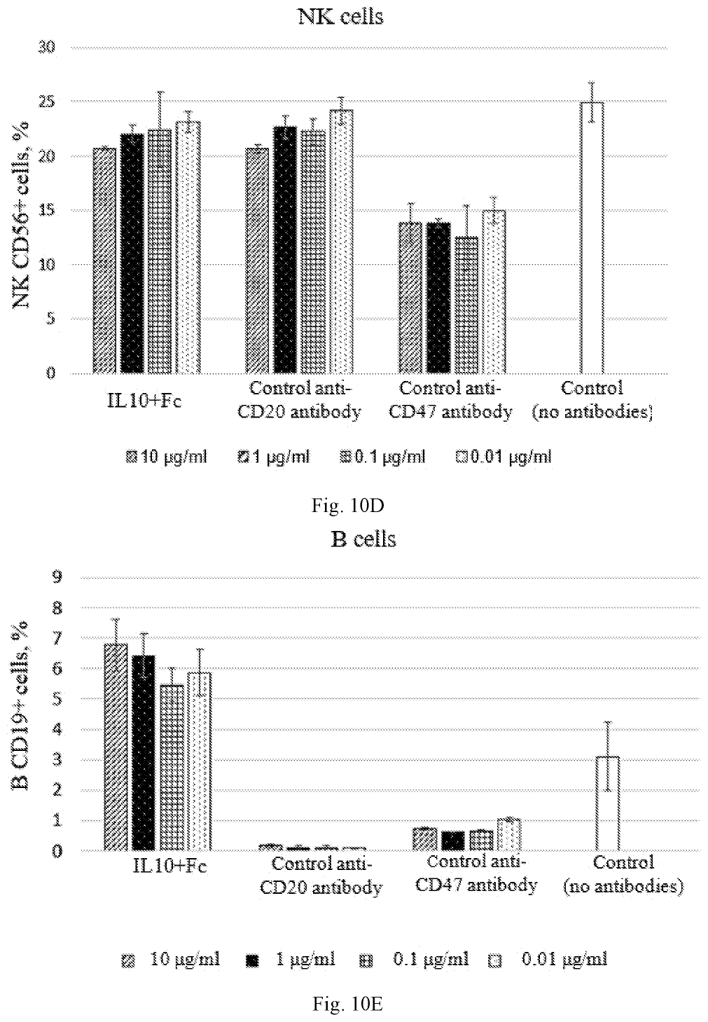

It has been shown that IL-10+Fc of the invention does not exhibit antibody-dependent cellular cytotoxicity in the assay using the Jurkat-NFAT-Luc-CD16-V176 reporter cell line (FIG. 10).

Example 13

Measurement of autocytotoxicity.

The assay allows in vitro assessment of antibody-induced depletion of main subpopulations of white blood cells in the blood, which in turn makes it possible to assess the safety of the therapeutic molecules being developed even before in vivo studies.

The assay was performed in a 96-well culture plate for suspension cultures. The suspension contained per well 300,000 freshly isolated PBMCs from healthy donors and IL-10+Fc of the invention at the specified concentration, the final volume of the cell suspension in a well was 150 μl. Obinutuzumab (aCD20) and anti-CD47 antibody were used as a positive control. All suspension components were prepared in RPMI-1640 medium supplemented with 10% fetal bovine serum. After mixing PBMCs and antibodies, the plate was incubated for 16 h at 37° C., 5% CO₂. The proportion of CD45+, CD56+, CD19+, CD3+, CD4+and CD8+ subpopulations in PBMCs in suspensions was then measured by directly staining the suspensions with fluorescent-labeled antibodies against the corresponding CDs followed by analyzing the cells using a flow cytofluorometer. For the CD56+, CD19+, CD3+ cells, the graphs show the proportion thereof relative to CD45+ cells of test suspension, whereas for the CD4+, CD8+ cells, the graphs show the proportion thereof relative to CD45+CD3+ cells.

The in vitro autocytotoxicity assay did not show that IL-10+Fc of the invention induces significant depletion of the NK, B and T cell populations in human PBMCs (FIG. 11A, 11B, 11C, 11D, 11E). Further, the control antibody Obinutuzumab (aCD20) showed almost complete depletion of the CD20+B cell population, and the anti-CD47 antibody showed almost 50% depletion of NK cells.

Example 14

Study of antitumor activity in vivo.

The study was performed on Balb/c mice (males aged 4-6 weeks, with a body weight of 18-24 g). 0.1 ml CT26 tumor cell suspension was injected subcutaneously into the right lateral side of mice in an amount of 2*10⁵ cells. When the tumors reached an approximate volume (V=LW2/2) of 70 mm3, the animals were divided into groups such that the average tumor volume in the groups did not differ by more than 10%. All of the drugs were administered by intraperitoneal (i.p.) administration twice a week for 3 weeks. In all cases, IL-10-Fc of the invention was administered at a dose of 0.1 mg/kg, amPD-1 was administered at a dose of 10 mg/kg. In groups with the combination of IL-10-Fc of the invention, amPD-1 was administered on the day after the administration of IL-10-Fc of the invention. Animals from the “tumor growth control” group were administered with a buffer. The linear size of the tumor node was assessed twice a week. ITG (index of tumor growth) was calculated as the ratio of the tumor volume on the day of measurement (1-19) to the tumor volume on the day of treatment initiation.

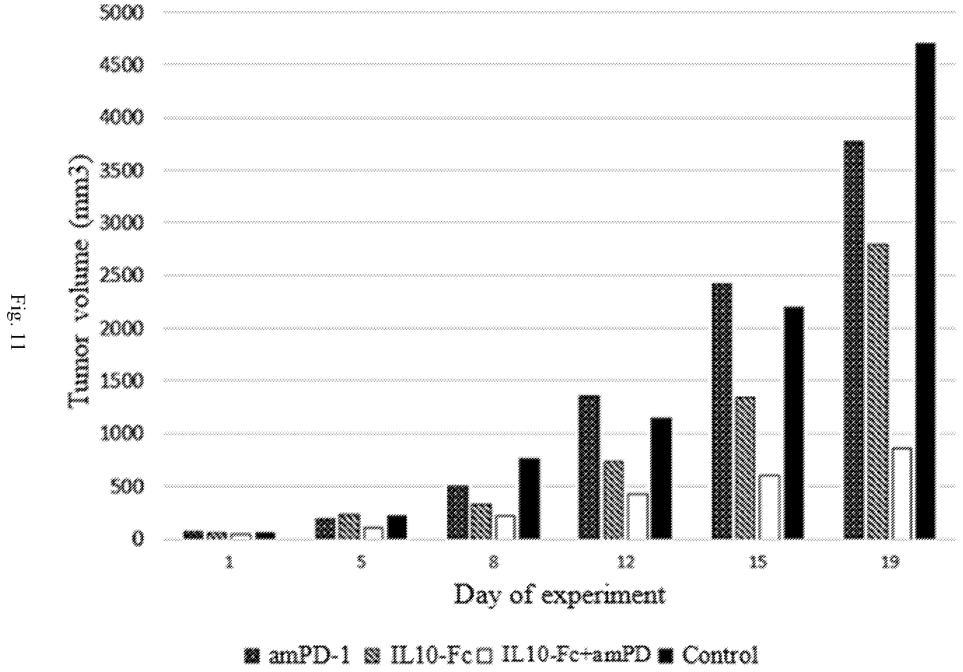

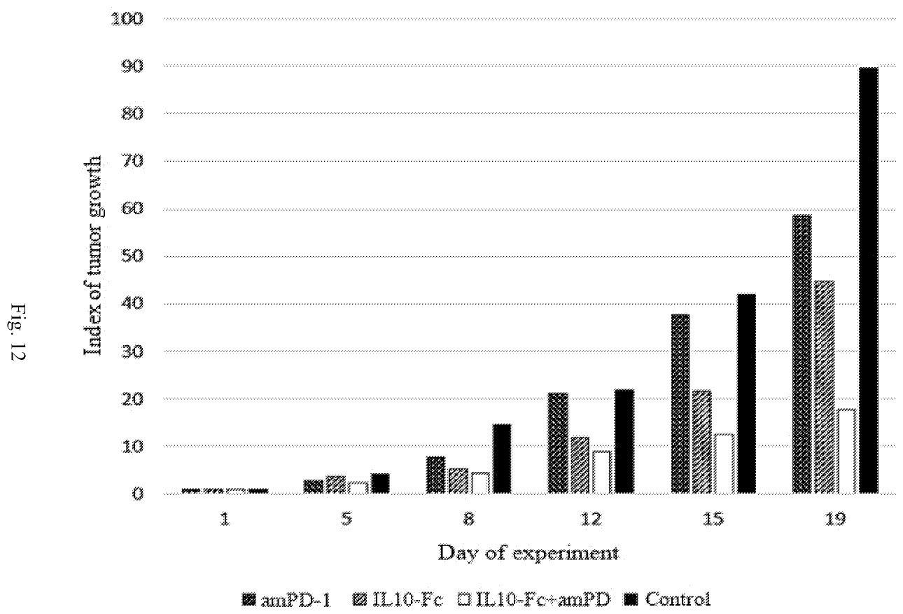

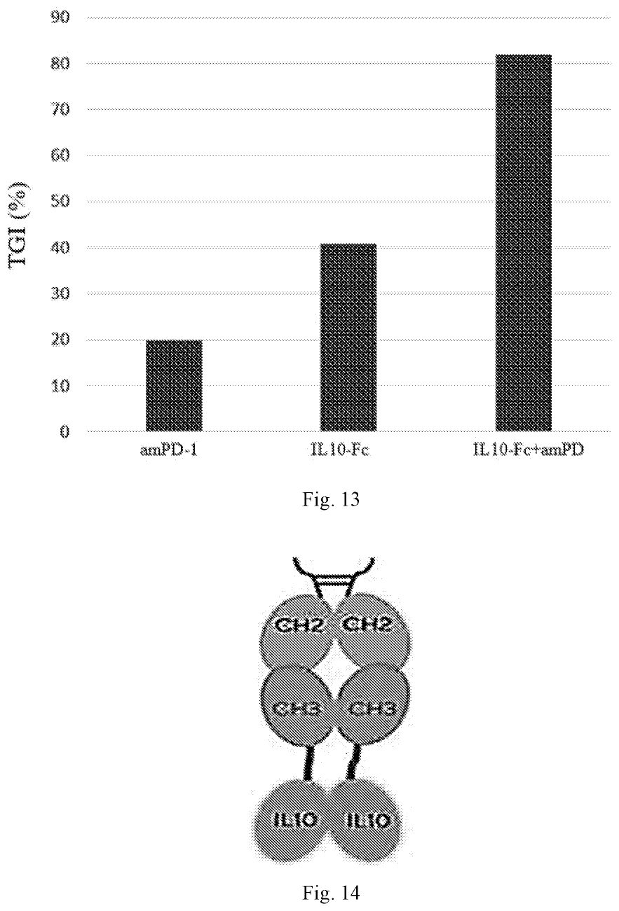

It has been shown that IL-10+Fc of the invention has antitumor activity in monotherapy, and, further, the combination of IL-10+Fc of the invention with anti-PD1 antibody has a pronounced synergy in antitumor activity as compared to monotherapy with either the immunocytokine IL-10+Fc of the invention or anti-PD-1 antibody (FIG. 12, 13, 14).

1. An isolated immunocytokine for activating the human IL-10Rα receptor, comprising a homodimeric complex based on IL-10 and human IgG1 Fc fragment, wherein the monomer based on IL-10 and human IgG1 Fc fragment comprises the amino acid sequence of SEQ ID NO:1.

2. An isolated nucleic acid, which encodes the immunocytokine as claimed in claim 1.

3. The isolated nucleic acid according to claim 2, wherein the nucleic acid is DNA.

4. The isolated nucleic acid according to claim 2, wherein the nucleic acid comprises a nucleotide sequence with SEQ ID NO:3.

5. The isolated nucleic acid according to claim 2, wherein the nucleic acid comprises a nucleotide sequence with SEQ ID NO:4.

6. An expression vector comprising the nucleic acid according to any of claims 2-5.

7. A method for producing a host cell for producing the immunocytokine according to claim 1, comprising transformation of the cell with the vector according to claim 6.

8. A host cell for producing the immunocytokine according to claim 1, comprising the nucleic acid according to any of claims 2-5.

9. A method for producing the immunocytokine according to claim 1, comprising culturing of the host cell according to claim 8 in a culture medium under conditions sufficient to produce said immunocytokine, if necessary, followed by isolation and purification of the resulting immunocytokine.

10. A pharmaceutical composition comprising the immunocytokine according to claim 1 and one or more pharmaceutically acceptable excipients.

11. A pharmaceutical composition according to claim 1, wherein said pharmaceutical composition is for activating the human IL-10Rα receptor.

12. The pharmaceutical composition according to claim 10 for treating an oncological disease.

13. The pharmaceutical composition according to claim a12, wherein the oncological disease is selected from the group comprising: HNSCC (head and neck squamous cell carcinoma), cervical cancer, cancer of unknown primary, glioblastoma, esophageal cancer, bladder cancer, TNBC (triple-negative breast cancer), CRC (colorectal cancer), hepatocellular carcinoma, melanoma, NSCLC (non-small cell lung cancer), kidney cancer, ovarian cancer, colorectal cancer with microsatellite instability, leukemia (acute leukemia or myeloblastic leukemia), lymphoma, multiple myeloma, breast cancer, prostate cancer, bladder cancer, sarcoma, hepatocellular carcinoma, glioblastoma, Hodgkin's lymphoma, T- and B-cell acute lymphoblastic leukemia, small cell lung cancer, refractory non-Hodgkin's B-cell lymphoma, follicular lymphoma, marginal zone B-cell lymphoma, diffuse large B-cell lymphoma, pancreatic cancer, ovarian cancer, higher-risk myelodysplastic syndrome.

14. A pharmaceutical composition for activating the human IL-10Rα receptor, comprising the immunocytokine according to claim 1 and at least one other therapeutically active compound.

15. The pharmaceutical composition according to claim 14 for treating an oncological disease.

16. The pharmaceutical composition according to claim 15, wherein the oncological disease is selected from the group comprising: HNSCC (head and neck squamous cell carcinoma), cervical cancer, cancer of unknown primary, glioblastoma, esophageal cancer, bladder cancer, TNBC (triple-negative breast cancer), CRC (colorectal cancer), hepatocellular carcinoma, melanoma, NSCLC (non-small cell lung cancer), kidney cancer, ovarian cancer, colorectal cancer with microsatellite instability, leukemia (acute leukemia or myeloblastic leukemia), lymphoma, multiple myeloma, breast cancer, prostate cancer, bladder cancer, sarcoma, hepatocellular carcinoma, glioblastoma, Hodgkin's lymphoma, T- and B-cell acute lymphoblastic leukemia, small cell lung cancer, refractory non-Hodgkin's B-cell lymphoma, follicular lymphoma, marginal zone B-cell lymphoma, diffuse large B-cell lymphoma, pancreatic cancer, ovarian cancer, higher-risk myelodysplastic syndrome.

17. The pharmaceutical composition according to any of claims 14-15, wherein the other therapeutically active compound is an antibody, chemotherapeutic agent or hormone therapy agent.

18. The pharmaceutical composition according to any of claims 14-15, wherein the other therapeutically active compound is an immune checkpoint inhibitor.

19. The pharmaceutical composition according to claim 18, wherein the immune checkpoint inhibitor is selected from an antibody that specifically binds to PD-1, an antibody that specifically binds to PD-L1or an antibody that specifically binds to CTLA-4.

20. The pharmaceutical composition according to claim 19, wherein the antibody that specifically binds to PD-1 is selected from the group comprising: prolgolimab, pembrolizumab, nivolumab.

21. The pharmaceutical composition according to claim 19, wherein the antibody that specifically binds to CTLA-4 is ipilimumab.

22. The use according to claim 19, wherein the antibody that specifically binds to PD-L1 and is selected from the group comprising: durvalumab, avelumab, atezolizumab, manelimab.

23. A method of treating an oncological disease, comprising administering to a subject the immunocytokine according to claim 1 or the pharmaceutical composition according to any of claims 10, 14, in need of such treatment, in a therapeutically effective amount.

24. The method for treating according to claim 23, wherein the oncological disease is selected from the group comprising: HNSCC (head and neck squamous cell carcinoma), cervical cancer, cancer of unknown primary, glioblastoma, esophageal cancer, bladder cancer, TNBC (triple-negative breast cancer), CRC (colorectal cancer), hepatocellular carcinoma, melanoma, NSCLC (non-small cell lung cancer), kidney cancer, ovarian cancer, colorectal cancer with microsatellite instability, leukemia (acute leukemia or myeloblastic leukemia), lymphoma, multiple myeloma, breast cancer, prostate cancer, bladder cancer, sarcoma, hepatocellular carcinoma, glioblastoma, Hodgkin's lymphoma, T- and B-cell acute lymphoblastic leukemia, small cell lung cancer, refractory non-Hodgkin's B-cell lymphoma, follicular lymphoma, marginal zone B-cell lymphoma, diffuse large B-cell lymphoma, pancreatic cancer, ovarian cancer, higher-risk myelodysplastic syndrome.

25. A method for activating the human IL-10Rα receptor in a subject in need of such activation, comprising administering to a subject an effective amount of the immunocytokine according to claim 1 or the pharmaceutical composition as according to any of claims 10, 14.

26. The use of the immunocytokine according to claim 1 or the pharmaceutical composition according to any of claims 10, 14 for treating, in a subject in need of such treatment, an oncological disease.

27. The use according to claim 26, wherein the oncological disease is selected from the group comprising: HNSCC (head and neck squamous cell carcinoma), cervical cancer, cancer of unknown primary, glioblastoma, esophageal cancer, bladder cancer, TNBC (triple-negative breast cancer), CRC (colorectal cancer), hepatocellular carcinoma, melanoma, NSCLC (non-small cell lung cancer), kidney cancer, ovarian cancer, colorectal cancer with microsatellite instability, leukemia (acute leukemia or myeloblastic leukemia), lymphoma, multiple myeloma, breast cancer, prostate cancer, bladder cancer, sarcoma, hepatocellular carcinoma, glioblastoma, Hodgkin's lymphoma, T- and B-cell acute lymphoblastic leukemia, small cell lung cancer, refractory non-Hodgkin's B-cell lymphoma, follicular lymphoma, marginal zone B-cell lymphoma, diffuse large B-cell lymphoma, pancreatic cancer, ovarian cancer, higher-risk myelodysplastic syndrome.

28. The use of the immunocytokine according to claim 1 or at least one other therapeutically active compound for the treatment, in a subject in need of such treatment, of an oncological disease.

29. The use according to claim 28, wherein the oncological disease is selected from the group comprising: HNSCC (head and neck squamous cell carcinoma), cervical cancer, cancer of unknown primary, glioblastoma, esophageal cancer, bladder cancer, TNBC (triple-negative breast cancer), CRC (colorectal cancer), hepatocellular carcinoma, melanoma, NSCLC (non-small cell lung cancer), kidney cancer, ovarian cancer, colorectal cancer with microsatellite instability, leukemia (acute leukemia or myeloblastic leukemia), lymphoma, multiple myeloma, breast cancer, prostate cancer, bladder cancer, sarcoma, hepatocellular carcinoma, glioblastoma, Hodgkin's lymphoma, T- and B-cell acute lymphoblastic leukemia, small cell lung cancer, refractory non-Hodgkin's B-cell lymphoma, follicular lymphoma, marginal zone B-cell lymphoma, diffuse large B-cell lymphoma, pancreatic cancer, ovarian cancer, higher-risk myelodysplastic syndrome.

30. The use according to any of claims 28-29, wherein the other therapeutically active compound is an antibody, chemotherapeutic agent or hormone therapy agent.

31. The use according to any of claims 28-29, wherein the other therapeutically active compound is an immune checkpoint inhibitor.

32. The use according to claim 31, wherein the immune checkpoint inhibitor is selected from an antibody that specifically binds to PD-1, an antibody that specifically binds to PD-L1 or an antibody that specifically binds to CTLA-4.

33. The use according to claim 32, wherein the antibody that specifically binds to PD-1 is selected from the group comprising: prolgolimab, pembrolizumab, nivolumab.

34. The use according to claim 32, wherein the antibody that specifically binds to CTLA-4 is ipilimumab.

35. The use according to claim 32, wherein the antibody that specifically binds to PD-L1 and is selected from the group comprising: durvalumab, avelumab, atezolizumab, manelimab.
